SEQUENCE LISTING (50 sequences)

SEQ ID NO: 1 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDNLAARDFINWLIQTKITK

SEQ ID NO: 2 - protein, 33 aa, synthetic construct; identical to SEQ ID NO: 1
  REGION 1..33: note = triple agonists
  MOD_RES 2: note = MISC_FEATURE - X = Aib

SEQ ID NO: 3 - protein, 33 aa, synthetic construct
  REGION 1..33: note = triple agonists
  MOD_RES 2: note = MISC_FEATURE - X = Aib
HXDGTFISDYATILDNLAARDFINWLIQTKITK

SEQ ID NO: 4 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDLLAARDFINWLIQTKITK

SEQ ID NO: 5 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDALAARDFINWLIQTKITK

SEQ ID NO: 6 - protein, 33 aa, synthetic construct; identical to SEQ ID NO: 1
  REGION 1..33: note = triple agonists
  MOD_RES 2: note = MISC_FEATURE - X = Aib
  MOD_RES 33: note = AMIDATION

SEQ ID NO: 7 - protein, 33 aa, synthetic construct
  REGION 1..33: note = triple agonists
  MOD_RES 2: note = MISC_FEATURE - X = Aib
  MOD_RES 2: note = AMIDATION
HXDGTFISDYSTILENLAARDFINWLIQTKITK

SEQ ID NO: 8 - protein, 33 aa, synthetic construct
  REGION 1..33: note = triple agonists
  MOD_RES 2: note = MISC_FEATURE - X = Aib
  MOD_RES 2: note = AMIDATION
HXDGTFISDYSTILDNLAAREFINWLIQTKITK

SEQ ID NO: 9 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDNLAARDFIEWLIQTKITK

SEQ ID NO: 10 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSIILDNLAARDFINWLIQTKITK

SEQ ID NO: 11 - protein, 33 aa, synthetic construct
  REGION 1..33: note = triple agonists
  MOD_RES 2: note = MISC_FEATURE - X = Aib
  MOD_RES 33: note = AMIDATION
HXEGTFISDYSTILDNLAARDFINWLIQTKITK

SEQ ID NO: 12 - protein, 33 aa, synthetic construct
  REGION 1..33: note = triple agonists
  MOD_RES 2: note = MISC_FEATURE - X = Aib
  MOD_RES 13: note = MISC_FEATURE - X = Aib
  MOD_RES 33: note = AMIDATION
HXDGTFISDYSTXLDNLAARDFINWLIQTKITK

SEQ ID NO: 13 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTELDNLAARDFINWLIQTKITK

SEQ ID NO: 14 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDNLAARDFINWLIQTKGKK

SEQ ID NO: 15 - protein, 33 aa, synthetic construct
  REGION 1..33: note = triple agonists
  MOD_RES 2: note = MISC_FEATURE - X = Aib
  MOD_RES 33: note = AMIDATION
HXDGTFISDYSTILDNIAARDFINWLIQTKITK

SEQ ID NO: 16 - protein, 33 aa, synthetic construct
  REGION 1..33: note = triple agonists
  MOD_RES 2: note = MISC_FEATURE - X = Aib
  MOD_RES 33: note = AMIDATION
YXDGTFISDYSTILDNLAARDFINWLIQTKITK

SEQ ID NO: 17 - protein, 33 aa, synthetic construct
  REGION 1..33: note = triple agonists
  PEPTIDE 1..33: note = peptide
HADGTFTSDYSTILDNLAAKDFINWLIQTKGKK

SEQ ID NO: 18 - protein, 33 aa, synthetic construct
  REGION 1..33: note = triple agonists
  PEPTIDE 1..33: note = peptide
HADGTFTSDYSTILDNLAARDFINWLIQTKGKK

SEQ ID NO: 19 - protein, 42 aa, synthetic construct
  REGION 1..42: note = triple agonists
  PEPTIDE 1..42: note = peptide
HADGTFTSDYSIILDKLAARDFINWLIQTKGKKNDWKHNITQ

SEQ ID NO: 20 - protein, 42 aa, synthetic construct
  REGION 1..42: note = triple agonists
  PEPTIDE 1..42: note = peptide
HADGTFTSDYSTILDKLAARDFINWLIQTKGKKNDWKHNITQ

SEQ ID NO: 21 - protein, 42 aa, synthetic construct
  REGION 1..42: note = triple agonists
  MOD_RES 13: note = MISC_FEATURE - X = Aib
HADGTFTSDYSTXLDKLAARDFINWLIQTKGKKNDWKHNITQ

SEQ ID NO: 22 - protein, 42 aa, synthetic construct
  REGION 1..42: note = triple agonists
  PEPTIDE 1..42: note = peptide
HADGTFTSDYSTILDKKAARDFINWLIQTKGKKNDWKHNITQ

SEQ ID NO: 23 - protein, 42 aa, synthetic construct; identical to SEQ ID NO: 20
  REGION 1..42: note = triple agonists
  PEPTIDE 1..42: note = peptide

SEQ ID NO: 24 - protein, 42 aa, synthetic construct; identical to SEQ ID NO: 20
  REGION 1..42: note = triple agonists
  PEPTIDE 1..42: note = peptide

SEQ ID NO: 25 - protein, 43 aa, synthetic construct
  REGION 1..43: note = triple agonists
  PEPTIDE 1..43: note = peptide
HADGTFTSDYSTILDKLAARDFINWLIQTKGKKNDWKHNITQK

SEQ ID NO: 26 - protein, 43 aa, synthetic construct
  REGION 1..43: note = triple agonists
  MOD_RES 13: note = MISC_FEATURE - X = Aib
HADGTFTSDYSTXLDKLAARDFINWLIQTKGKKNDWKHNITQK

SEQ ID NO: 27 - protein, 33 aa, synthetic construct; identical to SEQ ID NO: 5
  REGION 1..33: note = triple agonists
  MOD_RES 2: note = MISC_FEATURE - X = Aib

SEQ ID NO: 28 - protein, 33 aa, synthetic construct
  REGION 1..33: note = triple agonists
  MOD_RES 2: note = MISC_FEATURE - X = Aib
HXDGTFISDYKTILDNLAARDFINWLIQTKITK

SEQ ID NO: 29 - protein, 10 aa, synthetic construct
  REGION 1..10: note = triple agonists
  PEPTIDE 1..10: note = peptide
NDWKHNITQK

SEQ ID NO: 30 - protein, 4 aa, synthetic construct
  REGION 1..4: note = triple agonists
  PEPTIDE 1..4: note = peptide
NDWK

SEQ ID NO: 31 - protein, 5 aa, synthetic construct
  REGION 1..5: note = triple agonists
  PEPTIDE 1..5: note = peptide
NDWKH

SEQ ID NO: 32 - protein, 6 aa, synthetic construct
  REGION 1..6: note = triple agonists
  PEPTIDE 1..6: note = peptide
NDWKHN

SEQ ID NO: 33 - protein, 7 aa, synthetic construct
  REGION 1..7: note = triple agonists
  PEPTIDE 1..7: note = peptide
NDWKHNI

SEQ ID NO: 34 - protein, 8 aa, synthetic construct
  REGION 1..8: note = triple agonists
  PEPTIDE 1..8: note = peptide
NDWKHNIT

SEQ ID NO: 35 - protein, 9 aa, synthetic construct
  REGION 1..9: note = triple agonists
  PEPTIDE 1..9: note = peptide
NDWKHNITQ

SEQ ID NO: 36 - protein, 9 aa, synthetic construct
  REGION 1..9: note = triple agonists
  PEPTIDE 1..9
AEWKHAITQ

SEQ ID NO: 37 - protein, 42 aa, synthetic construct
  REGION 1..42: note = triple agonists
  REGION 1..42: note = MISC_FEATURE - hGIP
YAEGTFISDYSIAMDKIHQQDFVNWLLAQKGKKNDWKHNITQ

SEQ ID NO: 38 - protein, 37 aa, synthetic construct
  REGION 1..37: note = triple agonists
  REGION 1..37: note = MISC_FEATURE - hGLP-1(1-37)
HDEFERHAEGTFTSDVSSYLEGQAAKEFIAWLVKGRG

SEQ ID NO: 39 - protein, 30 aa, synthetic construct
  REGION 1..30: note = triple agonists
  REGION 1..30: note = MISC_FEATURE - hGLP-1(7-36)
HAEGTFTSDVSSYLEGQAAKEFIAWLVKGR

SEQ ID NO: 40 - protein, 31 aa, synthetic construct
  REGION 1..31: note = triple agonists
  REGION 1..31: note = MISC_FEATURE - hGLP-1(7-37)
HAEGTFTSDVSSYLEGQAAKEFIAWLVKGRG

SEQ ID NO: 41 - protein, 33 aa, synthetic construct
  REGION 1..33: note = triple agonists
  REGION 1..33: note = MISC_FEATURE - hGLP-2
HADGSFSDEMNTILDNLAARDFINWLIQTKITD

SEQ ID NO: 42 - protein, 34 aa, synthetic construct
  REGION 1..33: note = triple agonists
  VAR_SEQ 3: note = MISC_FEATURE - X = A or Aib
  VAR_SEQ 4: note = MISC_FEATURE - X = D or E
  VAR_SEQ 8: note = MISC_FEATURE - X = I, S or T
  VAR_SEQ 11: note = MISC_FEATURE - X = S or A
  VAR_SEQ 13: note = MISC_FEATURE - X = T or I
  VAR_SEQ 14: note = MISC_FEATURE - X = I, Aib or E
  VAR_SEQ 16: note = MISC_FEATURE - X = D or E
  VAR_SEQ 17: note = MISC_FEATURE - X = N, L, A, K or E
  VAR_SEQ 18: note = MISC_FEATURE - X = L, K or I
  VAR_SEQ 21: note = MISC_FEATURE - X = R or K
  VAR_SEQ 22: note = MISC_FEATURE - X = D, E or K
  VAR_SEQ 25: note = MISC_FEATURE - X = N or E
  VAR_SEQ 29: note = MISC_FEATURE - X = Q, A or K
  VAR_SEQ 30: note = MISC_FEATURE - X = T, Q or K
  VAR_SEQ 31: note = MISC_FEATURE - X = R or K
  VAR_SEQ 32: note = MISC_FEATURE - X = I or G
  VAR_SEQ 33: note = MISC_FEATURE - X = T or K
  VAR_SEQ 1: note = X = H or Y
XHXXGTFXSDYXXXLXXXAAXXFIXWLIXXXXXK

SEQ ID NO: 43 - protein, 34 aa, synthetic construct; identical to SEQ ID NO: 42
  REGION 1..33: note = triple agonists
  MOD_RES 3: note = MISC_FEATURE - X = Aib
  VAR_SEQ 4: note = MISC_FEATURE - X = D or E
  VAR_SEQ 8: note = MISC_FEATURE - X = I, S or T
  VAR_SEQ 12: note = MISC_FEATURE - X = S or A
  VAR_SEQ 13: note = MISC_FEATURE - X = T or I
  VAR_SEQ 14: note = MISC_FEATURE - X = I, Aib or E
  VAR_SEQ 16: note = MISC_FEATURE - X = D or E
  VAR_SEQ 17: note = MISC_FEATURE - X = N, L, A, K or E
  VAR_SEQ 18: note = MISC_FEATURE - X = L, K or I
  VAR_SEQ 21: note = MISC_FEATURE - X = R or K
  VAR_SEQ 22: note = MISC_FEATURE - X = D, E or K
  VAR_SEQ 25: note = MISC_FEATURE - X = N or E
  VAR_SEQ 29: note = MISC_FEATURE - X = Q, A or K
  VAR_SEQ 30: note = MISC_FEATURE - X = T, Q or K
  VAR_SEQ 31: note = MISC_FEATURE - X = R or K
  VAR_SEQ 32: note = MISC_FEATURE - X = I or G
  VAR_SEQ 33: note = MISC_FEATURE - X = T or K
  VAR_SEQ 1: note = X = H or Y

SEQ ID NO: 44 - protein, 34 aa, synthetic construct; identical to SEQ ID NO: 42
  REGION 1..33: note = triple agonists
  VAR_SEQ 3: note = MISC_FEATURE - X = A or Aib
  VAR_SEQ 4: note = MISC_FEATURE - X = D or E
  VAR_SEQ 8: note = MISC_FEATURE - X = S or T
  VAR_SEQ 12: note = MISC_FEATURE - X = S or A
  VAR_SEQ 13: note = MISC_FEATURE - X =T or I
  VAR_SEQ 14: note = MISC_FEATURE - X = I, Aib or E
  VAR_SEQ 16: note = MISC_FEATURE - X = D or E
  VAR_SEQ 17: note = MISC_FEATURE - X = N, L A, K or E
  VAR_SEQ 18: note = MISC_FEATURE - X = L, K or I
  VAR_SEQ 21: note = MISC_FEATURE - X = R or K
  VAR_SEQ 22: note = MISC_FEATURE - X = D, E or K
  VAR_SEQ 25: note = MISC_FEATURE - X = N or E
  VAR_SEQ 29: note = MISC_FEATURE - X = Q, A or K
  VAR_SEQ 30: note = MISC_FEATURE - X = T, Q or K
  VAR_SEQ 31: note = MISC_FEATURE - X = R or K
  VAR_SEQ 32: note = MISC_FEATURE - X = I or G
  VAR_SEQ 33: note = MISC_FEATURE - X = T or K
  VAR_SEQ 1: note = X = H or Y

SEQ ID NO: 45 - protein, 35 aa, synthetic construct
HADGTFISDYSTILDKLAARDFINWLIQTKGKKOH

SEQ ID NO: 46 - protein, 35 aa, synthetic construct
  MOD_RES 13: note = X = Aib
HADGTFISDYSTXLDKLAARDFINWLIQTKGKKOH

SEQ ID NO: 47 - protein, 35 aa, synthetic construct
HADGTFISDYSTILDNKAARDFINWLIQTKGKKOH

SEQ ID NO: 48 - protein, 35 aa, synthetic construct
HADGTFISDYSTILDNLAARDFINWLIQTKGKKOH

SEQ ID NO: 49 - protein, 36 aa, synthetic construct
HADGTFISDYSTILDNLAARDFINWLIQTKGKKKOH

SEQ ID NO: 50 - protein, 36 aa, synthetic construct
  MOD_RES 13: note = X = Aib
HADGTFISDYSTXLDNLAARDFINWLIQTKGKKKOH

(19) United States
(12) Patent Application Publication
(10) Pub. No.: US 2025/0304644 A1
(43) Pub. Date: Oct. 2, 2025

(54) PEPTIDES TRIPLE AGONISTS OF GIP, GLP-1 AND GLP-2

(71) Applicant: BAINAN BIOTECH APS, København N (DK)

(72) Inventors: Ditte RIBER, Brønshøj (DK); Mette Marie ROSENKILDE, Hellerup (DK)

(21) Appl. No.: 18/718,719

(22) PCT Filed: Dec. 16, 2022
(86) PCT No.: PCT/EP2022/086376

§ 371 Date: Jun. 11, 2024

(30) Foreign Application Priority Data
Dec. 17, 2021 (EP) 21215550.1

(51) Int. Cl. C07K 14/605; A61K 38/00

(52) U.S. Cl. CPC C07K 14/605; A61K 38/00

(57) ABSTRACT

The present invention relates to peptide triple agonists of the GIPR (glucose-dependent insulinotropic polypeptide receptor), the GLP-1R (glucagon-like peptide-1 receptor) and the GLP-2R (glucagon-like peptide-2 receptor), and their use in medicine.

### CROSS-REFERENCE TO RELATED APPLICATIONS

This application claims priority to co-pending PCT International Application Serial No. PCT/EP2022/086376 filed Dec. 16, 2022, which claims priority to European Patent Application No. 21215550.1 filed on Dec. 17, 2021, the entire content of both of which is incorporated herein by reference.

### SEQUENCE LISTING

The instant application contains a Sequence Listing which has been submitted electronically as an XML file format and is hereby incorporated by reference in its entirety. Said .XML file was created on Dec. 16, 2024, is named Amended sequence listing BAIN0002PA.xml and is 103,956 bytes in size.

### TECHNICAL FIELD

The present invention relates to peptide triple agonists of the GIPR (glucose-dependent insulinotropic polypeptide receptor), the GLP-1R (glucagon-like peptide-1 receptor) and the GLP-2R (glucagon-like peptide-2 receptor), and their use in medicine.

### BACKGROUND

Gastrointestinal peptides and adipokines are critical signalling molecules involved in controlling whole-body energy homeostasis. These circulating hormones regulate a variety of biological responses such as hunger, satiety and glucose uptake. In vivo experiments have established that these hormones also regulate bone metabolism, while associations between these hormones and bone mass have been observed in human clinical studies.

Incretins are gastrointestinal hormones that help to regulate carbohydrate metabolism in response to food intake. The two main incretins are glucose-dependent insulinotropic peptide (GIP) and glucagon-like peptide-1 (GLP-1), both secreted by intestinal epithelial cells. Intestinal glucagon-like peptide-2 (GLP-2) is co-secreted along with GLP-1 upon nutrient ingestion.

Gastrointestinal hormones released after meal ingestion, such as GIP and GLP-2 have been shown to regulate bone turnover; GIP has a positive effect on bone, and GLP-2 regulates bone homeostasis and has a positive contribution to bone mass.

GLP-1R may also be involved in bone metabolism.

Osteoporosis can be defined as a combination of reduced bone mass and altered bone quality, resulting in decreased bone strength with an increased risk of fractures. Gastrointestinal hormones including GIP and GLP-2 have each been implicated in bone metabolism and as potential therapies for treating osteoporosis.

WO 2018/069442 and WO 2020/169792 both disclose dual peptide agonists of GIPR and GLP-2R.

It is difficult to target specific receptors of the same receptor family, such as family B GPCR, while not targeting others. Potent peptides which are agonists of GIPR, GLP-1R and GLP-2R at the same time have so far not been disclosed.

### SUMMARY

Triple agonists that combine the properties of GIP, GLP-1 and GLP-2 receptor agonists are provided herewith. The present inventors have designed peptides triple agonists that target GIPR, GLP-1R and GLP-2R with unexpected high potency.

The triple GIPR, GLP-1R and GLP-2R agonists of the present invention induce less GIPR internalization compared to human GIP. This may result in prolonged activation of the GIPR and thus a more sustained effect. Moreover, some of the triple GIPR, GLP-1R and GLP-2R agonists of the present invention also induce less GLP-1R and/or GLP-2R internalization compared to human GLP-1 and/or GLP-2. Les internalization of any one of the GIPR, GLP-1R and/or GLP-2R may result in prolonged activation and thus a more sustained and greater effect.

One aspect of the present disclosure relates to peptide triple agonist comprising or consisting of the sequence

(SEQ ID NO: 42)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is A or Aib,
X₃ is D or E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
with the proviso that when X₇ is I, then X₂ is Aib, and when X₂ is A, then X₇ is S or T, wherein said peptide is modified by attaching at least one fatty acid molecule at one or more amino acid residues at any one of positions of the C-terminal region of SEQ ID NO: 42, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

Another aspect of the present disclosure relates to a peptide triple agonist according to any of the preceding claims for use as a medicament, such as for use in a method of inhibiting bone resorption and/or stimulating bone formation and/or for use in a method of treating a bone disorder and/or for use in a method of treating iodiopathic osteoporosis and/or for use in a method of treating osteoporosis in an individual suffering from Duchenne muscular dystrophy (DMD) or cerebral Palsy.

### DESCRIPTION OF DRAWINGS

FIG. 1: The overall potencies of the triple peptide agonists on the human GIPR, GLP-1R and GLP-2R are shown in FIG. 1 as a radar plot. Here, the compounds 20, 21, 22, 28, 30, 31,32, 33, 34, 35 are shown in terms of their potencies in Gs activation, measured as CAMP accumulation, depicted in a radar plot. The numbers refer to −p Log EC₅₀ values. The corresponding values on the human glucagon receptor (GCGR) are also depicted, but all are with potencies lower than 100 nM (−p Log EC₅₀ values <7). The outer rings correspond to higher potencies, the inner rings correspond to lower potencies.

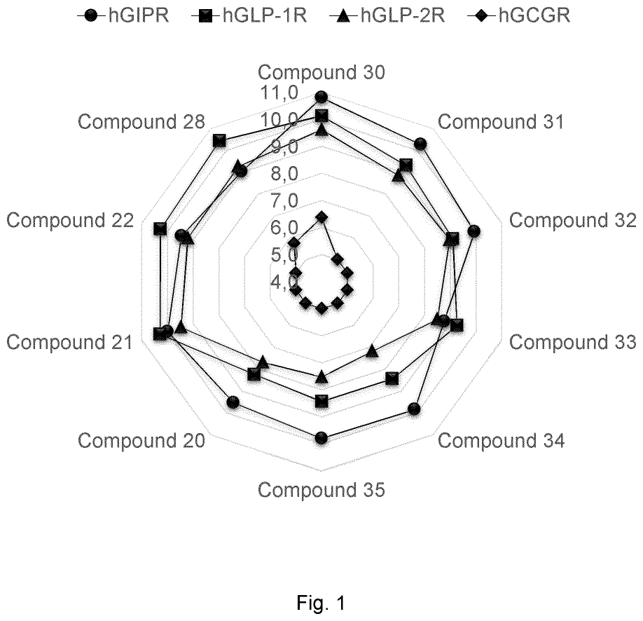

### DETAILED DESCRIPTION

### Definitions

The term “affinity” refers to the strength of binding between a ligand and its receptor.

The term “agonist” in the present context refers to a peptide as defined herein, capable of binding to and activating a receptor.

The term “triple agonist” refers to a peptide as defined herein, capable of binding to and activating at least three receptors, wherein the at least three receptors are different receptors. In the present context a triple agonist is an agonist of the GIPR, an agonist of the GLP-2R and an agonist of the GLP-1R. A triple agonist defined herewith may also have agonist activity towards additional receptors, whereby the triple agonist is an agonist of at least GIPR, GLP-2R and GLP-1R.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822 (b)(2) belong to the group of amino acids listed herewith: Y, G, F, M, A, S, I, L, T, V, P, K, H, Q, E, W, R, D, N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” as applied herein, one amino acid may be substituted for another, in one embodiment, within the groups of amino acids indicated herein below:

Amino acids having polar side chains (Asp, Glu, Lys, Arg, His, Asn, Gln, Ser, Thr, Pro, and Cys); Amino acids having non-polar side chains (Gly, Ala, Val, Leu, Ile, Phe, Trp, Tyr and Met); Amino acids having aliphatic side chains (Gly, Ala Val, Leu, Ile); Amino acids having cyclic side chains (Trp, His, Pro); Amino acids having aromatic side chains (Phe, Tyr, Trp); Amino acids having acidic, such as negatively charged side chains (Asp, Glu); Amino acids having basic, such as positively charged side chains (Lys, Arg, His); Amino acids having amide side chains (Asn, Gln); Amino acids having hydroxy side chains (Ser, Thr); Amino acids having sulphur-containing side chains (Cys, Met); Neutral, weakly hydrophobic amino acids (Pro, Ala, Gly, Ser, Thr); Hydrophilic, acidic amino acids (Gln, Asn, Glu, Asp); and Hydrophobic amino acids (Leu, Ile, Val).

Where the L or D form (optical isomers) has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

A “functional variant” of a peptide is a peptide capable of performing essentially the same functions as the peptide it is a functional variant of. In particular, a functional variant can bind the same molecules, preferably with the same affinity, as the peptide it is a functional variant of.

A “bioactive agent” (i.e. a biologically active substance/agent) is any agent, drug, compound, composition of matter or mixture which provides some pharmacologic, often beneficial, effect that can be demonstrated in vivo or in vitro. It refers to the peptide sequences defined herewith, compounds or compositions comprising these and nucleic acid constructs encoding said peptides. As used herein, this term further includes any physiologically or pharmacologically active substance that produces a localized or systemic effect in an individual. A ‘bioactive agent’ as used herein denotes collectively a peptide, a nucleic acid construct encoding said peptide, and a composition comprising a peptide.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “individual” refers to vertebrates, particular members of the mammalian species, preferably primates including humans. As used herein, ‘subject’ and ‘individual’ may be used interchangeably. Treatment of animals, such as mice, rats, dogs, cats, cows, horses, sheep and pigs, is, however, also within the scope of the present invention.

An “individual in need thereof” refers to an individual who may benefit from treatment. In one embodiment, said individual in need thereof is a diseased individual, wherein said disease may be a bone disorder.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

A treatment according to the invention can be prophylactic, ameliorating and/or curative.

“Pharmacologically effective amount”, “pharmaceutically effective amount” or “physiologically effective amount” of a “bioactive agent” is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered.

“Co-administering” or “co-administration” as used herein refers to the administration of one or more agonists and a state-of-the-art pharmaceutical composition. The at least two components can be administered separately, sequentially or simultaneously.

“Biased” as used herein in relation to the peptide triple agonists of the present disclosure refers to a peptide triple agonist being disproportioned relative to the cognate endogenous peptide hormones towards one (or more) of the three receptors GIPR, GLP-1R and GLP-2R, for example in relation to potency, activation, efficacy, and/or internalization. A biased peptide triple agonist of the present disclosure may be beneficial for certain applications.

GIP refers to glucose-dependent insulinotropic polypeptide, also known as Gastric Inhibitory Peptide (or polypeptide). As used herein the abbreviation hGIP is human GIP (Uniprot accession number P09681). GIP is derived from a 153-amino acid proprotein and circulates as a biologically active 42-amino acid peptide (positions 52-93). It is synthesized by K cells of the mucosa of the duodenum and the jejunum of the gastrointestinal tract.

Under physiological conditions the 42 amino acid hormone, GIP, is degraded by the enzyme dipeptidylpeptidase 4 (DPP-4), which cleaves at the third position of the GIP molecule to yield GIP3-42. GIP1-30 is produced as a result of post-translational processing. If GIP1-30 is secreted into the circulation in humans, the cleavage catalyzed by DPP-4 would result in GIP3-30.

The sequence of hGIP is:

(SEQ ID NO: 37)
YAEGTFISDYSIAMDKIHQQDFVNWLLAQKGKKNDWKHNITQ.

GIPR (or GIP receptor) refers to glucose-dependent insulinotropic polypeptide receptor(s). These seven-transmembrane proteins are found at least on beta-cells in the pancreas. As used herein the abbreviation hGIPR is human GIPR (Uniprot accession number P48546).

Several physiological effects of GIP have been identified. GIP is secreted from enteroendocrine K cells following nutrient intake and induces insulin secretion. The amount of insulin secreted is greater when glucose is administered orally than intravenously. GIP is also thought to have significant effects on fatty acid metabolism through stimulation of lipoprotein lipase activity in adipocytes. GIP recently appeared as a major player in bone remodelling, and deficiency in GIP receptors has been associated with a dramatic decrease in bone quality and a subsequent increase in fracture risk.

Glucagon-like peptide-2 (GLP-2) is a 33 amino acid peptide in humans created by specific post-translational proteolytic cleavage of proglucagon in a process that also liberates the related glucagon-like peptide-1 (GLP-1) and glucagon itself. GLP-2 is produced by the intestinal endocrine L cell and by various neurons in the central nervous system. Intestinal GLP-2 is co-secreted along with GLP-1 upon nutrient ingestion. When externally administered, GLP-2 produces a number of effects in humans and rodents, including intestinal growth, enhancement of intestinal function, reduction in bone breakdown and neuroprotection. GLP-2 and related analogs have potential as treatments for short bowel syndrome, Crohn's disease, osteoporosis and as adjuvant therapy during cancer chemotherapy.

The sequence of hGLP-2 is:

(SEQ ID NO: 41)
HADGSFSDEMNTILDNLAARDFINWLIQTKITD

The GLP-2 receptor (GLP-2R) is a G protein-coupled receptor superfamily member. GLP-2R is expressed in the gut and is closely related to the glucagon receptor (GCGR) and the GLP1 receptor (GLP-1R). As used herein the abbreviation hGLP-2R is human GLP-2R (e.g. Uniprot accession number 095838). As used herein the abbreviation hGLP-2 is human GLP-2 (Uniprot accession number not available). As used herein the abbreviation hGLP-1R is human GLP-1R (GLP-1 receptor) (e.g. Uniprot accession number P43220).

Glucagon-like peptide-1 (GLP-1) derives from the tissue-specific posttranslational processing of proglucagon. It is produced and secreted by intestinal enteroendocrine L cells and certain neurons within the nucleus of the solitary tract in the brainstem upon food consumption. In the intestinal L cells proglucagon is processed to C-terminally amidated GLP-1 (7-36) and small amounts C-terminally glycine-extended GLP-1 (7-37) and released in response to meal/glucose ingestion. Active GLP-1 composes two a-helices from amino acid position 13-20 and 24-35 separated by a linker region.

GLP-1 decreases blood sugar levels in a glucose-dependent manner by enhancing the secretion of insulin. Beside the insulinotropic effects, GLP-1 has been associated with numerous regulatory and protective effects. The action of GLP-1 is preserved in patients with type 2 diabetes and substantial pharmaceutical research has therefore been directed towards the development of GLP-1-based treatment. Endogenous GLP-1 is rapidly degraded primarily by dipeptidyl peptidase-4 (DPP-4), but also neutral endopeptidase 24.11 (NEP 24.11) and renal clearance, resulting in a half-life of approximately 2 minutes. Consequently, only 10-15% of GLP-1 reaches circulation intact, leading to fasting plasma levels of only 0-15 pmol/L. To overcome this, combination with DPP-4 inhibitors and the development degradation resistant variants of GLP-1 have been employed.

The sequences of native hGLP-1 are:

hGLP-1(1-37):
(SEQ ID NO: 38)
HDEFERHAEGTFTSDVSSYLEGQAAKEFIAWLVKGRG
hGLP-1(7-36):
(SEQ ID NO: 39)
HAEGTFTSDVSSYLEGQAAKEFIAWLVKGR
hGLP-1(7-37):
(SEQ ID NO: 40)
HAEGTFTSDVSSYLEGQAAKEFIAWLVKGRG

GIPR, GLP-1R and GLP-2R peptide triple agonists The present inventors have designed novel peptides which are triple agonists of GIPR, GLP-1R and GLP-2R and have unexpected and unprecedented high potency. These peptides are based on the sequences of hGIP and hGLP-2. A triple agonist of the GIPR, GLP-1R and GLP-2R means that the peptide binds to and/or activates at least the GIPR, the GLP-1R and the GLP-2R. This makes them potentially useful in a range of therapeutic applications.

The peptides are designed by combining amino acids/amino acid stretches from the GIP peptide (SEQ ID NO: 37) and from the GLP-2 peptide (SEQ ID NO: 41). Amino acid residues/stretches from GLP-1 peptide (SEQ ID NO: 38) may also be present in the triple agonists of the present disclosure. These amino acids are preferably involved in one or more of receptor binding, receptor affinity, receptor activity and/or otherwise relevant for the agonist activity of the peptides. In some embodiments, the peptides are designed to comprise amino acid residues which are not derived from any of GIP peptide (SEQ ID NO: 37), GLP-1 peptide (SEQ ID NO: 38) and GLP-2 peptide (SEQ ID NO: 41), but which may render said peptides of the present disclosure more stable and/or more potent and/or more selective compared to a peptide dual agonist comprising residues deriving solely from GIP peptide (SEQ ID NO: 37) and GLP-2 peptide (SEQ ID NO: 41), or solely from GIP peptide (SEQ ID NO: 37), GLP-1 peptide (SEQ ID NO: 38) and GLP-2 peptide (SEQ ID NO: 41).

It is an aspect to provide a peptide triple agonist comprising or consisting of the sequence

(SEQ ID NO: 42)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is A or Aib,
X₃ is D or E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
with the proviso that when X₇ is I, then X₂ is Aib, and when X₂ is A, then X₇ is S or T, wherein said peptide is modified by attaching at least one fatty acid molecule at one or more amino acid residues at any one of positions of the C-terminal region of SEQ ID NO: 42, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

It is an aspect to provide a peptide triple agonist comprising or consisting of the sequence

(SEQ ID NO: 43)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is Aib,
X₃ is Dor E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
wherein said peptide is modified by attaching at least one fatty acid molecule at one or more amino acid residues at any one of positions of the C-terminal region of SEQ ID NO: 43, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

It is an aspect to provide a peptide triple agonist comprising or consisting of the sequence

(SEQ ID NO: 44)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is A or Aib,
X₃ is D or E,
X₇ is S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
wherein said peptide is modified by attaching at least one fatty acid molecule at one or more amino acid residues at any one of positions of the C-terminal region of SEQ ID NO: 44, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

The numbering in SEQ ID NO: 42, 43 and SEQ ID NO: 44 (such as SEQ ID NO: 42 to 44) corresponds to the numbering of the amino acid residues in said sequence, such that:
X₁ is the residue at position 1, X₂ is the residue at position 2, X₃ is the residue at position 3, G is the residue at position 4, T is the residue at position 5, F is the residue at position 6, X₇ is the residue at position 7, S is the residue at position 8, D is the residue at position 9, Y is the residue at position 10, X₁₁ is the residue at position 11, X₁₂ is the residue at position 12, X₁₃ is the residue at position 13, L is the residue at position 14, X₁₅ is the residue at position 15, X₁₆ is the residue at position 16, X₁₇ is the residue at position 17, A is the residue at position 18, A is the residue at position 19, X₂₀ is the residue at position 20, X₂₁ is the residue at position 21, F is the residue at position 22, I is the residue at position 23, X₂₄ is the residue at position 24, W is the residue at position 25, L is the residue at position 26, I is the residue at position 27, X₂₈ is the residue at position 28, X₂₉ is the residue at position 29, X₃₀ is the residue at position 30, X₃₁ is the residue at position 31, X₃₂ is the residue at position 32, K is the residue at position 33 of said SEQ ID NO: 42 to 44.

Similarly, J is a peptide that is numbered as the continuation of residues 1-33, thus the one or more amino acid residues of J (NDWKHNITQK; SEQ ID NO: 29) are for example numbered so that N is position 34, D is position 35, Wis position 36, K is position 37, H is position 38, N is position 39, I is position 40, T is position 41, Q is position 42, K is position 43.

However, in one embodiment J may consist of only one amino acid residue, and in that case said amino acid residue is position 34, for example even if said amino acid residue is not an N. For example, in one embodiment J may consist of only one K, and in that case said K is position 34.

The C-terminal region of the peptide triple agonists of the present disclosure such as of a peptide triple agonist of SEQ ID NO: 42 to 44 consists of the amino acid residues stretching from position 16 to the C-terminus of SEQ ID NO: 42 to 44. For example, the C-terminal region of the peptide triple agonists of the present disclosure such as of a peptide triple agonist of SEQ ID NO: 42 to 44 consists of the amino acid residues stretching from position 16 to position 33 of SEQ ID NO: 42 to 44, such as from position 16 to position 34 of SEQ ID NO: 42 to 44, such as from position 16 to position 42 of SEQ ID NO: 42 to 44, such as from position 16 to position 43 of SEQ ID NO: 42 to 44.

The C-terminal region of the peptide triple agonists of the present disclosure such as of a peptide triple agonist of SEQ ID NO: 42 to 44 consists of the amino acid residues 16 to 33 of SEQ ID NO: 42 to 44 and of the amino acid residues of J (SEQ ID NO: 29).

Thus, in one embodiment, the peptide triple agonist of the present disclosure is modified by attaching at least one fatty acid molecule at one or more amino acid residues at any one of positions 16 to 33 of SEQ ID NO: 42 to 44 and/or at any one of positions of J (SEQ ID NO: 29).

Also, in one embodiment, the peptide triple agonist of the present disclosure is modified by attaching at least one fatty acid molecule at one or more amino acid residues at any one of positions 16 to 43 of SEQ ID NO: 42 to 44.

In one embodiment, the peptide triple agonist of the present disclosure is modified by attaching at least one fatty acid molecule at one or more amino acid residues at any one of positions 16 to 33 of SEQ ID NO: 42 to 44.

In one embodiment, the present disclosure relates to a peptide triple agonist of GIPR, GLP-1R and GLP-2R comprising the amino acid sequence SEQ ID NO: 42 to 44, or a functional variant thereof, or a functional fragment thereof.

SEQ ID NO: 42 to 44 may also be written as (H/Y)(A/Aib)(D/E)GTF(I/S/T)SDY(S/A)(I/T)(E/Aib/I)L(D/E)(E/L/K/A/N)(L/K/I)AA(R/K)(D/E/K)FI(N/E)WLI(Q/A/K)(T/Q/K)(R/K)(I/G)(T/K)K-J; or a functional variant thereof, or a functional fragment thereof.

Said peptide triple agonist being an agonist of GIPR, an agonist of GLP-1R and an agonist of GLP-2R implies that the peptide triple agonist is an agonist of GIPR, GLP-1R and of GLP-2R. It does not exclude that the peptide triple agonist can bind and/or activate further receptors. However, the peptide triple agonists of the present disclosure are designed to activate with high potency the GIPR, GLP-1R and GLP-2R.

The present inventors have in fact found that attachment of a fatty acid at the C-terminal region of a peptide triple agonist of the present disclosure, such as at anyone of the amino acid residues at positions 16 to 43 of a peptide of SEQ ID NO: 42 to 44, results in said peptide having ability to bind and/or activate with high potency the GIPR, GLP-1R and GLP-2R.

Reference to GIPR, GLP-2R, and GLP-1R as used herein throughout can be substituted with hGIPR, hGLP-2R, and hGLP-1R.

In one embodiment the peptide triple agonist comprises
a. amino acids from hGLP-2 (SEQ ID NO: 41) and/or hGIP (SEQ ID NO:37) at the N-terminus,
b. a central/N-terminal stretch of amino acids primarily from hGIP (SEQ ID NO: 37),
c. a central/C-terminal stretch of amino acids primarily from hGLP-2 (SEQ ID NO: 41), and
d. optionally amino acids primarily from hGIP (SEQ ID NO: 37) at the C-terminus.

In one embodiment amino acids at positions 12 to 20 or 12 to 30 are primarily from hGLP-2 (SEQ ID NO: 41).

In one embodiment amino acids at positions 5 to 11 or 7 to 11 are primarily from hGIP (SEQ ID NO: 37).

In one embodiment amino acids at positions 7 and/or 8 are primarily from hGLP-1 (SEQ ID NO: 38). Presence of these residues may increase GLP-1R potency.

In one embodiment when amino acids are defined as “primarily from” this means that at least 50% of the amino acids are from the peptide in question, such as at least 55%, such as at least 60%, such as at least 65%, such as at least 70%, such as at least 75%, such as at least 80%, such as at least 85%, such as at least 90%, such as at least 95%, such as 100% are from the peptide in question (i.e. from hGIP, from hGLP-1 or from hGLP-2).

In one embodiment when amino acids are defined as “primarily from” this means that 50-55% of the amino acids are from the peptide in question, such as 55-60%, such as 60-65%, such as 65-70%, such as 70-75%, such as 75-80%, such as 80-85%, such as 85-90%, such as 90-95%, such as 95-100% are from the peptide in question (i.e. from hGIP, from hGLP-1 or from hGLP-2).

When an amino acid is from hGIP or from hGLP-2, this means that the amino acid at a certain position of the peptide triple agonist corresponds to the amino acid at the same position of hGIP or hGLP-2. For instance, if the amino acid at position 1 is from GIP this means that the amino acid is “Y”. If the amino acid at position 1 is from GLP-2 this means that the amino acid is “H”.

In one embodiment the peptide triple agonist comprises or consists of 20 to 43 consecutive amino acids; such as 20 to 22 amino acids, such as 22 to 24 amino acids, such as 24 to 26 amino acids, such as 26 to 28 amino acids, such as 28 to 30 amino acids, such as 30 to 32 amino acids, such as 32 to 34 amino acids, such as 34 to 36 amino acids, such as 36 to 38 amino acids, such as 38 to 40 amino acids, such as 40 to 42, such as 38 to 43 consecutive amino acids as defined herein.

In one embodiment the peptide triple agonist comprises or consists of 20 to 43 consecutive amino acids; such as 20, 21, 22, 23, 24, 25, 26, 27, 28, 29, 30, 31, 32, 33, 34, 35, 36, 37, 38, 39, 40, 41, 42 or 43 consecutive amino acids.

In one embodiment the peptide triple agonist comprises or consists of 20 to 33 consecutive amino acids, such as 20-21, such as 21-22, such as 22-23, such as 23-24, such as 24-25, such as 25-26, such as 26-27, such as 27-28, such as 28-29, such as 29-30, such as 30-31, such as 31-32, such as 32-33, such as 33-34, such as 34-35, such as 35-36, such as 36-37, such as 37-38, such as 38-39, such as 39-40, such as 40-41, such as 41-42, such as 42-43 consecutive amino acids.

In one embodiment the peptide triple agonist of the present disclosure comprises or consists of 33 consecutive amino acids.

In one embodiment the peptide triple agonist of the present disclosure comprises or consists of 34 consecutive amino acids.

In one embodiment the peptide triple agonist of the present disclosure comprises or consists of 42 consecutive amino acids.

In one embodiment the peptide triple agonist of the present disclosure comprises or consists of 43 consecutive amino acids.

In one embodiment, presence of Tyrosine (Y) at position 1 results in a peptide triple agonist which has increased potency towards GIPR. In one embodiment, presence of Tyrosine (Y) at position 1 results in a peptide triple agonist which has reduced potency towards GLP-1R.

In one embodiment the peptide triple agonist of the present disclosure comprises or consists of the amino acid sequence SEQ ID NO: 42-44, wherein X₂ is Aib, and wherein said peptide is modified by attaching at least one fatty acid molecule at the C-terminus of SEQ ID NO: 42-44.

In one embodiment the peptide triple agonist of the present disclosure comprises or consists of the amino acid sequence SEQ ID NO: 42-44, wherein X₂ is Aib, and wherein said peptide is modified by attaching at least one fatty acid molecule at the amino acid residue at position 33 of SEQ ID NO: 42-44.

In one embodiment the peptide triple agonist of the present disclosure comprises or consists of the amino acid sequence SEQ ID NO: 42-44, wherein X₂ is Aib, and wherein said peptide is modified by attaching at least one fatty acid molecule at the amino acid residue at position 34 of SEQ ID NO: 42-44.

In one embodiment the peptide triple agonist of the present disclosure comprises or consists of the amino acid sequence SEQ ID NO: 42-44, wherein X₂ is Aib, and wherein said peptide is modified by attaching at least one fatty acid molecule at the amino acid residue at position 42 of SEQ ID NO: 42-44.

In one embodiment the peptide triple agonist of the present disclosure comprises or consists of the amino acid sequence SEQ ID NO: 42-44, wherein X₂ is Aib, and wherein said peptide is modified by attaching at least one fatty acid molecule at the amino acid residue at position 43 of SEQ ID NO: 42-44.

In one embodiment, presence of Serine(S) in position 11, .e, wherein X₁₁ is S, results in a peptide triple agonist which has increased potency towards GLP-2R, thus a triple agonist biased for GLP-2R.

In one embodiment the peptide triple agonist of the present disclosure comprises or consists of the amino acid sequence SEQ ID NO: 42-44, wherein X₁₆ is N, L, A, or E and
wherein said peptide is modified by attaching at least one fatty acid molecule at the C-terminus of SEQ ID NO: 42-44.

In one embodiment the peptide triple agonist of the present disclosure comprises or consists of the amino acid sequence SEQ ID NO: 42-44, wherein X₁₆ is N, L, A, or E and
wherein said peptide is modified by attaching at least one fatty acid molecule at the amino acid residue at position 33 of SEQ ID NO: 42-44.

In one embodiment the peptide triple agonist of the present disclosure comprises or consists of the amino acid sequence SEQ ID NO: 42-44, wherein X₁₆ is N, L, A, or E and wherein said peptide is modified by attaching at least one fatty acid molecule at the amino acid residue at position 34 of SEQ ID NO: 42-44.

In one embodiment the peptide triple agonist of the present disclosure comprises or consists of the amino acid sequence SEQ ID NO: 42-44, wherein X₁₆ is N, L, A, or E and wherein said peptide is modified by attaching at least one fatty acid molecule at the amino acid residue at position 42 of SEQ ID NO: 42-44.

In one embodiment the peptide triple agonist of the present disclosure comprises or consists of the amino acid sequence SEQ ID NO: 42-44, wherein X₁₆ is N, L, A, or E and wherein said peptide is modified by attaching at least one fatty acid molecule at the amino acid residue at position 43 of SEQ ID NO: 42-44.

In one embodiment the peptide triple agonist of the present disclosure comprises or consists of the amino acid sequence SEQ ID NO: 42-44, wherein X₁₇ is L, or I.

In one embodiment the peptide triple agonist of the present disclosure comprises or consists of the amino acid sequence SEQ ID NO: 42-44, wherein X₂₀ is R.

In one embodiment the peptide triple agonist of the present disclosure comprises or consists of the amino acid sequence SEQ ID NO: 42-44, wherein X₇ is T. Presence of T at position 7, such as substitution of the I at position 7 of hGIP with a T, results in improved potency towards the GLP-1R.

In one embodiment the peptide triple agonist of the present disclosure comprises or consists of the amino acid sequence SEQ ID NO: 42-44, wherein X₇ is S. Presence of S at position 7, such as substitution of the I at position 7 of hGIP with a S, results in improved potency towards the GLP-1R.

In one embodiment the peptide triple agonist of the present disclosure comprises or consists of the amino acid sequence SEQ ID NO: 42-44, wherein X₁₃ is Aib. Thus, in one embodiment, Alanine at position 13 of hGIP, or Isoleucine at position 13 of hGLP-2 are substituted with 2-Aminoisobutyric acid. Presence of Aib at position 13 increases potency on at least one of GIPR, GLP-1R and GLP-2R.

In general, presence of one or more Aib residues makes the 3D structure of the peptide triple agonists of the present disclosure more rigid, which may improve potency and selectivity on GIPR, GLP-1R and/or GLP-2R. In one embodiment, presence of Aib in position 2 correlates with higher reduction of GIPR internalization i.e. reduced GIPR internalization. In one embodiment, presence of Aib in position 13 correlates with higher reduction of GIPR internalization i.e. reduced GIPR internalization. In one embodiment, peptide triple agonists of the present disclosure comprising Aib in position 2 and/or 13 are biased towards reduced internalization of GIPR.

In one embodiment the peptide triple agonist of the present disclosure comprises or consists of the amino acid sequence SEQ ID NO: 42-44, wherein X₁₆ is N, A or K. In one embodiment, presence of Alanine (A) in position 16, i.e. wherein X₁₆ is A, results in a peptide triple agonist which has increased activity towards GIPR.

In one embodiment the peptide triple agonist of the present disclosure comprises or consists of the amino acid sequence SEQ ID NO: 42-44, wherein X₂₈ is Q.

In one embodiment the peptide triple agonist of the present disclosure comprises or consists of the amino acid sequence SEQ ID NO: 42-44, wherein X₂₉ is T.

In one embodiment the peptide triple agonist of the present disclosure comprises or consists of the amino acid sequence SEQ ID NO: 42-44, wherein X₃₀ is K.

In one embodiment the peptide triple agonist of the present disclosure comprises or consists of the amino acid sequence SEQ ID NO: 42-44, wherein X₂ is Aib, X₃₁ is I, and X₃₂ is T.

In one embodiment the peptide triple agonist of the present disclosure comprises or consists of the amino acid sequence SEQ ID NO: 42-44, wherein X₂ is Aib, X₃₁ is G, and X₃₂ is K.

In one embodiment the peptide triple agonist of the present disclosure comprises or consists of the amino acid sequence SEQ ID NO: 42-44, wherein X₇ is T or S, X₃₁ is G, and X₃₂ is K.

In one embodiment the peptide triple agonist of the present disclosure comprises or consists of the amino acid sequence SEQ ID NO: 42-44, wherein X₇ is T, X₃₁ is G, and X₃₂ is K.

In one embodiment the peptide triple agonist of the present disclosure comprises or consists of the amino acid sequence SEQ ID NO: 42-44, wherein the amino acid residue at position 33 is not a D. It was in fact found that substitution of the D at position 33 of GLP-2 with a K or another amino acid residue is advantageous and results in improved potency towards the GIPR and/or GLP-2R.

In one embodiment the peptide triple agonist of the present disclosure comprises or consists of the amino acid sequence SEQ ID NO: 42-44, wherein X₃₁X₃₂ is selected from the group consisting of IT, GK, GT, IK and IT. In one embodiment X₃₁X₃₂ is IT. In one embodiment X₃₁X₃₂ is GK.

In one embodiment the peptide triple agonist of the present disclosure comprises or consists of the amino acid sequence SEQ ID NO: 42-44, wherein J is omitted.

In one embodiment the peptide triple agonist of the present disclosure comprises or consists of the amino acid sequence SEQ ID 42-44, wherein J is selected from the group consisting of:
ND, NDW, KDW, NDW, NDK, NDWK, NDWKH, NDWKHN, NDWKHNI, NDWKHNIT, NDWKHNITQ, and AEWKHAITQ or a variant comprising one amino acid substitution.

In one embodiment the peptide triple agonist of the present disclosure comprises or consists of the amino acid sequence SEQ ID NO: 42-44, wherein J is K.

In one embodiment the peptide triple agonist of the present disclosure comprises or consists of the amino acid sequence SEQ ID NO: 42-44, wherein J is selected from the group consisting of NDWKHNITQ, NDWKHNITQK.

In one embodiment the peptide triple agonist of the present disclosure comprises an amino acid sequence selected from the group consisting of:

Compound 1:
(SEQ ID NO: 1)
H-Aib-DGTFISDYSTILDNLAARDFINWLIQTKITK-NH₂,
Compound 2:
(SEQ ID NO: 2)
H-Aib-DGTFISDYSTILDNLAARDFINWLIQTKITK-OH,
Compound 3:
(SEQ ID NO: 3)
H-Aib-DGTFISDYATILDNLAARDFINWLIQTKITK-OH,
Compound 4:
(SEQ ID NO: 4)
H-Aib-DGTFISDYSTILDLLAARDFINWLIQTKITK-NH₂,
Compound 5:
(SEQ ID NO: 5)
H-Aib-DGTFISDYSTILDALAARDFINWLIQTKITK-NH₂,
Compound 6:
(SEQ ID NO: 6)
H-Aib-DGTFISDYSTILDNLAARDFINWLIQTKITK-NH₂,
Compound 7:
(SEQ ID NO: 7)
H-Aib-DGTFISDYSTILENLAARDFINWLIQTKITK-NH₂,
Compound 8:
(SEQ ID NO: 8)
H-Aib-DGTFISDYSTILDNLAAREFINWLIQTKITK-NH₂,
Compound 9:
(SEQ ID NO: 9)
H-Aib-DGTFISDYSTILDNLAARDFIEWLIQTKITK-NH₂,
Compound 10:
(SEQ ID NO: 10)
H-Aib-DGTFISDYSIILDNLAARDFINWLIQTKITK-NH₂,
Compound 11:
(SEQ ID NO: 11)
H-Aib-EGTFISDYSTILDNLAARDFINWLIQTKITK-NH₂,
Compound 12:
(SEQ ID NO: 12)
H-Aib-DGTFISDYST-Aib-LDNLAARDFINWLIQTKITK-NH₂,
Compound 13:
(SEQ ID NO: 13)
H-Aib-DGTFISDYSTELDNLAARDFINWLIQTKITK-NH₂,
Compound 14:
(SEQ ID NO: 14)
H-Aib-DGTFISDYSTILDNLAARDFINWLIQTKGKK-NH₂,
Compound 15:
(SEQ ID NO: 15)
H-Aib-DGTFISDYSTILDNIAARDFINWLIQTKITK-NH₂,
Compound 16:
(SEQ ID NO: 16)
Y-Aib-DGTFISDYSTILDNLAARDFINWLIQTKITK-NH₂,
Compound 17:
(SEQ ID NO: 1)
H-Aib-DGTFISDYSTILDNLAARDFINWLIQTKITK-NH₂,
Compound 18:
(SEQ ID NO: 17)
HADGTFTSDYSTILDNLAAKDFINWLIQTKGKK-OH,
Compound 19:
(SEQ ID NO: 18)
HADGTFTSDYSTILDNLAARDFINWLIQTKGKK-OH,
Compound 20:
(SEQ ID NO: 19)
HADGTFTSDYSIILDKLAARDFINWLIQTKGKKNDWKHNITQ-OH,
Compound 21:
(SEQ ID NO: 20)
HADGTFTSDYSTILD-KLAARDFINWLIQTKGKKNDWKHNITQ-OH,
Compound 22:
(SEQ ID NO: 21)
HADGTFTSDYST-Aib-LDKLAARDFINWLIQTKGKKNDWKHNITQ-
OH,
Compound 23:
(SEQ ID NO: 22)
HADGTFTSDYSTILDKKAARDFINWLIQTKGKKNDWKHNITQ-OH,
Compound 24:
(SEQ ID NO: 23)
HADGTFTSDYSTILDKLAARDFINWLIQTKGKKNDWKHNITQ-OH,
Compound 25:
(SEQ ID NO: 24)
HADGTFTSDYSTILDKLAARDFINWLIQTKGKKNDWKHNITQ-OH,
Compound 26:
(SEQ ID NO: 25)
HADGTFTSDYSTILDKLAARDFINWLIQTKGKKNDWKHNITQK-OH,
Compound 27:
(SEQ ID NO: 26)
HADGTFTSDYST-Aib-LDKLAARDFINWLIQTKGKKNDWKHNITQK-
OH,
Compound 28:
(SEQ ID NO: 27)
H-Aib-DGTFISDYSTILDALAARDFINWLIQTKITK-OH,
(SEQ ID NO: 45)
HADGTFISDYSTILDKLAARDFINWLIQTKGKK-OH,
(SEQ ID NO: 46)
HADGTFISDYST-Aib-LDKLAARDFINWLIQTKGKK-OH,
(SEQ ID NO: 47)
HADGTFISDYSTILDNKAARDFINWLIQTKGKK-OH,
(SEQ ID NO: 48)
HADGTFISDYSTILDNLAARDFINWLIQTKGKK-OH,
(SEQ ID NO: 49)
HADGTFISDYSTILDNLAARDFINWLIQTKGKKK-OH,
and
(SEQ ID NO: 50)
HADGTFISDYST-Aib-LDNLAARDFINWLIQTKGKKK-OH,

or a functional variant thereof, wherein said functional variant comprises 1 or 2 individual amino acid substitutions.

In one aspect, the present disclosure relates to
a peptide triple agonist comprising or
consisting of an amino acid sequence selected
from the group consisting of:
Compound 1:
(SEQ ID NO: 1)
H-Aib-DGTFISDYSTILDNLAARDFINWLIQTKITK-NH₂,
Compound 2:
(SEQ ID NO: 2)
H-Aib-DGTFISDYSTILDNLAARDFINWLIQTKITK-OH,
Compound 3:
(SEQ ID NO: 3)
H-Aib-DGTFISDYATILDNLAARDFINWLIQTKITK-OH,
Compound 4:
(SEQ ID NO: 4)
H-Aib-DGTFISDYSTILDLLAARDFINWLIQTKITK-NH₂,
Compound 5:
(SEQ ID NO: 5)
H-Aib-DGTFISDYSTILDALAARDFINWLIQTKITK-NH₂,
Compound 6:
(SEQ ID NO: 6)
H-Aib-DGTFISDYSTILDNLAARDFINWLIQTKITK-NH₂,
Compound 7:
(SEQ ID NO: 7)
H-Aib-DGTFISDYSTILENLAARDFINWLIQTKITK-NH₂,
Compound 8:
(SEQ ID NO: 8)
H-Aib-DGTFISDYSTILDNLAAREFINWLIQTKITK-NH₂,
Compound 9:
(SEQ ID NO: 9)
H-Aib-DGTFISDYSTILDNLAARDFIEWLIQTKITK-NH₂,
Compound 10:
(SEQ ID NO: 10)
H-Aib-DGTFISDYSIILDNLAARDFINWLIQTKITK-NH₂,
Compound 11:
(SEQ ID NO: 11)
H-Aib-EGTFISDYSTILDNLAARDFINWLIQTKITK-NH₂,
Compound 12:
(SEQ ID NO: 12)
H-Aib-DGTFISDYST-Aib-LDNLAARDFINWLIQTKITK-NH₂,
Compound 13:
(SEQ ID NO: 13)
H-Aib-DGTFISDYSTELDNLAARDFINWLIQTKITK-NH₂,
Compound 14:
(SEQ ID NO: 14)
H-Aib-DGTFISDYSTILDNLAARDFINWLIQTKGKK-NH₂,
Compound 15:
(SEQ ID NO: 15)
H-Aib-DGTFISDYSTILDNIAARDFINWLIQTKITK-NH₂,
Compound 16:
(SEQ ID NO: 16)
Y-Aib-DGTFISDYSTILDNLAARDFINWLIQTKITK-NH₂,
Compound 17:
(SEQ ID NO: 1)
H-Aib-DGTFISDYSTILDNLAARDFINWLIQTKITK-NH₂,
Compound 18:
(SEQ ID NO: 17)
HADGTFTSDYSTILDNLAAKDFINWLIQTKGKK-OH,
Compound 19:
(SEQ ID NO: 18)
HADGTFTSDYSTILDNLAARDFINWLIQTKGKK-OH,
Compound 20:
(SEQ ID NO: 19)
HADGTFTSDYSIILDKLAARDFINWLIQTKGKKNDWKHNITQ-OH,
Compound 21:
(SEQ ID NO: 20)
HADGTFTSDYSTILD-KLAARDFINWLIQTKGKKNDWKHNITQ-OH,
Compound 22:
(SEQ ID NO: 21)
HADGTFTSDYST-Aib-LDKLAARDFINWLIQTKGKKNDWKHNITQ-
OH,
Compound 23:
(SEQ ID NO: 22)
HADGTFTSDYSTILDKKAARDFINWLIQTKGKKNDWKHNITQ-OH,
Compound 24:
(SEQ ID NO: 23)
HADGTFTSDYSTILDKLAARDFINWLIQTKGKKNDWKHNITQ-OH,
Compound 25:
(SEQ ID NO: 24)
HADGTFTSDYSTILDKLAARDFINWLIQTKGKKNDWKHNITQ-OH,
Compound 26:
(SEQ ID NO: 25)
HADGTFTSDYSTILDKLAARDFINWLIQTKGKKNDWKHNITQK-OH,
Compound 27:
(SEQ ID NO: 26)
HADGTFTSDYST-Aib-LDKLAARDFINWLIQTKGKKNDWKHNITQK-
OH,
Compound 28:
(SEQ ID NO: 27)
H-Aib-DGTFISDYSTILDALAARDFINWLIQTKITK-OH,
Compound 30: BB-153
HADGTFISDYSTILDKLAARDFINWLIQTKGKK-OH,
Compound 31: BB-154
HADGTFISDYST-Aib-LDKLAARDFINWLIQTKGKK-OH,
Compound 32: BB-155
HADGTFISDYSTILDNKAARDFINWLIQTKGKK-OH,
Compound 33: BB-157
HADGTFISDYSTILDNLAARDFINWLIQTKGKK-OH,
Compound 34: BB-159
HADGTFISDYSTILDNLAARDFINWLITKGKKK-OH,
Compound 35: BB-160
HADGTFISDYST-Aib-LDNLAARDFINWLIQTKGKKK-OH

or a functional variant thereof, wherein said functional variant comprises 1 to 3 individual amino acid substitutions,
wherein said peptide is modified by attaching at least one fatty acid molecule at one or more amino acid residues at any one of the positions of the C-terminal region of the above sequences, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

In one aspect, the present disclosure relates to a peptide triple agonist comprising or consisting of an amino acid sequence selected from the group consisting of:

Compound 1:
(SEQ ID NO: 1)
H-Aib-DGTFISDYSTILDNLAARDFINWLIQTKITK-NH₂,
Compound 2:
(SEQ ID NO: 2)
H-Aib-DGTFISDYSTILDNLAARDFINWLIQTKITK-OH,
Compound 3:
(SEQ ID NO: 3)
H-Aib-DGTFISDYATILDNLAARDFINWLIQTKITK-OH,
Compound 4:
(SEQ ID NO: 4)
H-Aib-DGTFISDYSTILDLLAARDFINWLIQTKITK-NH₂,
Compound 5:
(SEQ ID NO: 5)
H-Aib-DGTFISDYSTILDALAARDFINWLIQTKITK-NH₂,
Compound 6:
(SEQ ID NO: 6)
H-Aib-DGTFISDYSTILDNLAARDFINWLIQTKITK-NH₂,
Compound 7:
(SEQ ID NO: 7)
H-Aib-DGTFISDYSTILENLAARDFINWLIQTKITK-NH₂,
Compound 8:
(SEQ ID NO: 8)
H-Aib-DGTFISDYSTILDNLAAREFINWLIQTKITK-NH₂,
Compound 9:
(SEQ ID NO: 9)
H-Aib-DGTFISDYSTILDNLAARDFIEWLIQTKITK-NH₂,
Compound 10:
(SEQ ID NO: 10)
H-Aib-DGTFISDYSIILDNLAARDFINWLIQTKITK-NH₂,
Compound 11:
(SEQ ID NO: 11)
H-Aib-EGTFISDYSTILDNLAARDFINWLIQTKITK-NH₂,
Compound 12:
(SEQ ID NO: 12)
H-Aib-DGTFISDYST-Aib-LDNLAARDFINWLIQTKITK-NH₂,
Compound 13:
(SEQ ID NO: 13)
H-Aib-DGTFISDYSTELDNLAARDFINWLIQTKITK-NH₂,
Compound 14:
(SEQ ID NO: 14)
H-Aib-DGTFISDYSTILDNLAARDFINWLIQTKGKK-NH₂,
Compound 15:
(SEQ ID NO: 15)
H-Aib-DGTFISDYSTILDNIAARDFINWLIQTKITK-NH₂,
Compound 16:
(SEQ ID NO: 16)
Y-Aib-DGTFISDYSTILDNLAARDFINWLIQTKITK-NH₂,
Compound 17:
(SEQ ID NO: 1)
H-Aib-DGTFISDYSTILDNLAARDFINWLIQTKITK-NH₂,
Compound 18:
(SEQ ID NO: 17)
HADGTFTSDYSTILDNLAAKDFINWLIQTKGKK-OH,
Compound 19:
(SEQ ID NO: 18)
HADGTFTSDYSTILDNLAARDFINWLIQTKGKK-OH,
Compound 20:
(SEQ ID NO: 19)
HADGTFTSDYSIILDKLAARDFINWLIQTKGKKNDWKHNITQ-OH,
Compound 21:
(SEQ ID NO: 20)
HADGTFTSDYSTILD-KLAARDFINWLIQTKGKKNDWKHNITQ-OH,
Compound 22:
(SEQ ID NO: 21)
HADGTFTSDYST-Aib-LDKLAARDFINWLIQTKGKKNDWKHNITQ-
OH,
Compound 23:
(SEQ ID NO: 22)
HADGTFTSDYSTILDKKAARDFINWLIQTKGKKNDWKHNITQ-OH,
Compound 24:
(SEQ ID NO: 23)
HADGTFTSDYSTILDKLAARDFINWLIQTKGKKNDWKHNITQ-OH,
Compound 25:
(SEQ ID NO: 24)
HADGTFTSDYSTILDKLAARDFINWLIQTKGKKNDWKHNITQ-OH,
Compound 26:
(SEQ ID NO: 25)
HADGTFTSDYSTILDKLAARDFINWLIQTKGKKNDWKHNITQK-OH,
Compound 27:
(SEQ ID NO: 26)
HADGTFTSDYST-Aib-LDKLAARDFINWLIQTKGKKNDWKHNITQK-
OH,
Compound 28:
(SEQ ID NO: 27)
H-Aib-DGTFISDYSTILDALAARDFINWLIQTKITK-OH,
Compound 30: BB-153
HADGTFISDYSTILDKLAARDFINWLIQTKGKK-OH,
Compound 31: BB-154
HADGTFISDYST-Aib-LDKLAARDFINWLIQTKGKK-OH,
Compound 32: BB-155
HADGTFISDYSTILDNKAARDFINWLIQTKGKK-OH,
Compound 33: BB-157
HADGTFISDYSTILDNLAARDFINWLIQTKGKK-OH,
Compound 34: BB-159
HADGTFISDYSTILDNLAARDFINWLITKGKKK-OH,
Compound 35: BB-160
HADGTFISDYST-Aib-LDNLAARDFINWLIQTKGKKK-OH

or a functional variant thereof, wherein said functional variant comprises 1 to 3 individual amino acid substitutions, with the proviso that the amino acid residue at position 2 is Aib and/or the amino acid residue at position 7 is T or S,
wherein said peptide is modified by attaching at least one fatty acid molecule at one or more amino acid residues at any one of the positions of the C-terminal region of the above sequences, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

In one embodiment the peptide triple agonist according to the present disclosure is C-terminally amidated (—NH₂).

In one embodiment the C-terminus of the peptide triple agonist according to the present disclosure is a carboxylic acid.

In one embodiment a functional variant of a peptide triple agonist has at least 60% sequence identity, such as at least 70% sequence identity, such as at least 75% sequence identity, such as at least 80% sequence identity, such as at least 85% sequence identity, such as at least 90% sequence identity, such as at least 95% sequence identity, such as at least 97% sequence identity to said peptide triple agonist.

In one embodiment a functional variant of a peptide triple agonist has 60 to 65% sequence identity, such as 65 to 70% sequence identity, such as 70 to 75% sequence identity, such as 75 to 80% sequence identity, such as 80 to 85% sequence identity, such as 85 to 90% sequence identity, such as 90 to 95% sequence identity, such as 95 to 99% sequence identity, such as 99 to 100% sequence identity to said peptide triple agonist. ‘Identity’ and ‘sequence identity’ may be used interchangeably herein.

In one embodiment a functional variant comprises one or more amino acid substitutions, such as 1 to 8 amino acid substitutions, such as 1 to 2, 2 to 3, 3 to 4, 4 to 5, 5 to 6, 6 to 7 or 7 to 8 amino acid substitutions.

In one embodiment a functional variant comprises one amino acid substitution, two amino acid substitutions, three amino acid substitutions, four amino acid substitutions or five amino acid substitutions.

In one embodiment said amino acid substitutions are conservative amino acid substitutions. In one embodiment said functional variant comprises one or more conservative amino acid substitutions.

A conservative substitution (or synonymous substitution) is the substitution of amino acids whose side chains have similar biochemical properties and thus do not affect the function of the peptide.

The identity between amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90, or by simple comparison of the specific amino acids present at corresponding positions in two peptide sequences to be compared. Homology may be used as a synonym to identity/sequence identity.

Conservative substitutions may be introduced in any one or more positions of a peptide according to the present disclosure, as long as the variant remains functional. It may however also be desirable to introduce non-conservative substitutions in one or more positions (non-synonymous substitutions).

Any amino acids as defined herein may be in the L- or D-configuration. If nothing is specified, reference to the L-isomeric form is preferably meant.

The standard and/or non-standard amino acids may be linked by peptide bonds (to form a linear peptide chain), or by non-peptide bonds (e.g. via the variable side-chains of the amino acids). Preferably, the amino acids of the present disclosure are linked by peptide bonds.

The terms ‘peptide’ and ‘isolated peptide’ may be used interchangeably herein. The terms ‘variant’ and ‘functional variant’ may be used interchangeably herein. The terms ‘fragment’ and ‘functional fragment’ may be used interchangeably herein. When reference is made to a ‘peptide’ herewith, this term will encompass both references to a peptide per se, and also to a peptide for use as defined herein.

In one embodiment the peptide is non-naturally occurring.

In one embodiment the peptide is synthetic.

In one embodiment the peptide is an isolated peptide.

In one embodiment the peptide is a labeled peptide, such as radiolabeled or fluorescent labeled peptide.

In another embodiment, a variant as defined herein includes sequences wherein an alkyl amino acid is substituted for an alkyl amino acid, wherein an aromatic amino acid is substituted for an aromatic amino acid, wherein a sulfur-containing amino acid is substituted for a sulfur-containing amino acid, wherein a hydroxy-containing amino acid is substituted for a hydroxy-containing amino acid, wherein an acidic amino acid is substituted for an acidic amino acid, wherein a basic amino acid is substituted for a basic amino acid, and/or wherein a dibasic monocarboxylic amino acid is substituted for a dibasic monocarboxylic amino acid.

The term peptide also embraces post-translational modifications introduced by chemical or enzyme-catalyzed reactions, as are known in the art. These include acetylation, phosphorylation, methylation, glucosylation, glycation, amidation, hydroxylation, deimination, deamidation, carbamylation and sulfation of one or more amino acid residues, and also proteolytic modification by known proteinases including lysosomal kathepsins, and also calpains, secretases and matrix-metalloproteinases.

Also, functional equivalents of the peptides may comprise chemical modifications such as ubiquitination, labeling (e.g., with radionuclides, various enzymes, etc.), pegylation (derivatization with polyethylene glycol), or by insertion (or substitution by chemical synthesis) of amino acids such as ornithine, which do not normally occur in human proteins (non-proteinogenic).

Sterically similar compounds may be formulated to mimic the key portions of the peptide structure. This may be achieved by techniques of modelling and chemical designing known to those of skill in the art. For example, esterification and other alkylations may be employed to modify the amino terminus of e.g. a di-arginine peptide backbone, to mimic a tetra peptide structure. It will be understood that all such sterically similar constructs fall within the scope of the present invention. Peptides with N-terminal and C-terminal alkylations and esterifications are also encompassed within the present invention.

A contiguous or consecutive peptide sequence is a sequence of consecutive amino acids being linked linearly by peptide bonds. Contiguous and consecutive amino acid sequence is used interchangeably herein.

### Attachment of at Least One Fatty Acid Molecule

The peptide triple agonists of the present disclosure are characterized by being modified by attachment of at least one fatty acid molecule at an amino acid residues in the C-terminal region of the disclosed amino acid sequences. In fact, attachment of a fatty acid on the C-terminal region of the disclosed amino acid sequences, such as at any one of positions 16 to 43 of the disclosed sequences, results in a triple peptide agonist of hGIPR, hGLP-1R and hGLP-2R, which has high potency towards one or more of hGIPR, hGLP-1R and hGLP-2R.

In one embodiment the peptide triple agonist of the present disclosure is modified by attaching at least one fatty acid molecule at a Lysine (K) at any one of the positions of the C-terminal region of SEQ ID NO: 42, 43 and SEQ ID NO: 44 (such as SEQ ID NO: 42 to 44).

In one embodiment the peptide triple agonist of the present disclosure is modified by attaching at least one fatty acid molecule at any one of position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, positon 39, position 40, position 41, position 42 or position 43 of SEQ ID NO: 42 to 44.

In one embodiment the peptide triple agonist of the present disclosure is modified by attaching at least one fatty acid molecule at any one of position 16, position 17, position 20, position 33, position 34, position 37 or position 43 of SEQ ID NO: 42 to 44.

In one embodiment the peptide triple agonist of the present disclosure is modified by attaching at least one fatty acid molecule at one or more amino acid residues at any one of positions 16, 17, 20 to 43 of SEQ ID NO: 42 to 44.

In one embodiment the peptide triple agonist of the present disclosure is modified by attaching at least one fatty acid molecule at one or more amino acid residues at position 16 of SEQ ID NO: 42 to 44. Attaching a fatty acid in position 16 appears to be particular suitable for obtaining GIPR/GLP-2R/GLP-1R triple agonists with high potency towards all three receptors.

In one embodiment the peptide triple agonist of the present disclosure is modified by attaching at least one fatty acid molecule at one or more amino acid residues at position 17 of SEQ ID NO: 42 to 44.

In one embodiment the peptide triple agonist of the present disclosure is modified by attaching at least one fatty acid molecule at one or more amino acid residues at position 20 of SEQ ID NO: 42 to 44.

In one embodiment the peptide triple agonist of the present disclosure is modified by attaching at least one fatty acid molecule at one or more amino acid residues at position 43 of SEQ ID NO: 42 to 44.

In one embodiment the peptide triple agonist of the present disclosure is modified by attaching at least one fatty acid molecule at the C-terminus of SEQ ID NO: 42 to 44.

In one embodiment, attachment of one or more fatty acid at the C-terminus on residue 33 or 34 of a peptide triple agonist of the present disclosure results in a peptide triple agonist with reduced activation of GLP-2R. In contrast, in one embodiment, attachment of one or more fatty acid at the C-terminus on residue 42 or 43 of a peptide triple agonist of the present disclosure results in a peptide triple agonist with high activation of GLP-2R.

In one embodiment of the present disclosure, the fatty acid molecule is a straight-chain fatty acid.

In one embodiment of the present disclosure, said fatty acid molecule is a branched fatty acid.

In one embodiment of the present disclosure, said fatty acid molecule is a monoacyl fatty acid molecule, comprising one fatty acid.

In one embodiment of the present disclosure, said fatty acid molecule is a diacyl fatty acid molecule.

In one embodiment of the present disclosure, said fatty acid molecule comprises an acyl group selected from the group consisting of CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20).

In one embodiment of the present disclosure, said fatty acid molecule comprises two acyl groups individually selected from the group consisting of HOOC—CH₃(CH₂)₈CO— (decanoyl, C10), HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), and HOOC—CH₃(CH₂)₁₉CO— (19-carboxy-nonadecanoyl, C21).

In one embodiment said fatty acid molecule is C16 (palmitoyl).

In one embodiment of the present disclosure, said fatty acid molecule is attached to an amino acid residue directly, such as without the presence of a spacer or linker.

In one embodiment of the present disclosure, said fatty acid molecule is attached to an amino acid residue via a spacer or linker.

In one embodiment the spacer is a hydrophilic linker. In one embodiment the spacer is a non-natural amino acid hydrophilic linker.

In one embodiment the spacer is a repeat of individual spacer moieties. In one embodiment the spacer is a repeat of identical spacer moieties. In one embodiment the spacer is a repeat of different spacer moieties.

In one embodiment, the fatty acid molecule is attached to an amino acid residue via a spacer in such a way that a carboxyl group of the spacer forms an amide bond with an amino group of the fatty acid molecule.

In one embodiment the spacer comprises one or more moieties individually selected from the group consisting of:
one or more amino acids selected from the group consisting of succinic acid, Lys, Glu, Asp,
4-Abu,
y-aminobuturic acid,
one or more of γ-aminobutanoyl (γ-aminobutyric acid), γ-glutamyl (γ-glutamic acid), β-asparagyl, β-alanyl and glycyl, and
[γ-glutamic acid-8-amino-3,6-dioxaoctanoic acid]ₙ (γGlu-AEEAcₙ,), wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50.

In one embodiment of the present disclosure, the spacer or linker comprises yGlu.

In one embodiment of the present disclosure, said spacer comprises or consists of yGlu or yGlu-yGlu.

In some embodiments of the present disclosure the peptide triple agonists disclosed herein are acylated, which in some embodiments increase half-life and in vivo stability, in addition to rendering the peptide triple agonists selective agonists of GIPR and GLP-2R, and not GLP-1R, and retaining their agonistic potency.

In one embodiment the peptide triple agonist of the present disclosure is selected from the group consisting of

(SEQ ID NO: 1)
H-Aib-DGTFISDYSTILDNLAARDFINWLIQTKIT-K(C10)-NH₂,
(SEQ ID NO: 2)
H-Aib-DGTFISDYSTILDNLAARDFINWLIQTKIT-K(C10)-OH,
(SEQ ID NO: 3)
H-Aib-DGTFISDYATILDNLAARDFINWLIQTKIT-K(C10)-OH,
(SEQ ID NO: 4)
H-Aib-DGTFISDYSTILDLLAARDFINWLIQTKIT-K(C10)-NH₂,
(SEQ ID NO: 5)
H-Aib-DGTFISDYSTILDALAARDFINWLIQTKIT-K(C10)-NH₂,
(SEQ ID NO: 6)
H-Aib-DGTFISDYSTILDNLAARDFINWLIQTKIT-K(C10)-NH₂,
(SEQ ID NO: 7)
H-Aib-DGTFISDYSTILENLAARDFINWLIQTKIT-K(C10)-NH₂,
(SEQ ID NO: 8)
H-Aib-DGTFISDYSTILDNLAAREFINWLIQTKIT-K(C10)-NH₂,
(SEQ ID NO: 9)
H-Aib-DGTFISDYSTILDNLAARDFIEWLIQTKIT-K(C10)-NH₂,
(SEQ ID NO: 10)
H-Aib-DGTFISDYSIILDNLAARDFINWLIQTKIT-K(C10)-NH₂,
(SEQ ID NO: 11)
H-Aib-EGTFISDYSTILDNLAARDFINWLIQTKIT-K(C10)-NH₂,
(SEQ ID NO: 12)
H-Aib-DGTFISDYST-Aib-LDNLAARDFINWLIQTKIT-K(C10)-NH₂,
(SEQ ID NO: 13)
H-Aib-DGTFISDYSTELDNLAARDFINWLIQTKIT-K(C10)-NH₂,
(SEQ ID NO: 14)
H-Aib-DGTFISDYSTILDNLAARDFINWLIQTKGK-K(C10)-NH₂,
(SEQ ID NO: 15)
H-Aib-DGTFISDYSTILDNIAARDFINWLIQTKIT-K(C10)-NH₂,
(SEQ ID NO: 16)
Y-Aib-DGTFISDYSTILDNLAARDFINWLIQTKIT-K(C10)-NH₂,
(SEQ ID NO: 1)
H-Aib-DGTFISDYSTILDNLAARDFINWLIQTKIT-K(γGlu-C10)-NH₂,
(SEQ ID NO: 17)
HADGTFTSDYSTILDNLAA-K(2xAEEA-γGlu-C18-diacid)-DFINWLIQTKGKK-OH,
(SEQ ID NO: 18)
HADGTFTSDYSTILDNLAARDFINWLIQTKGK-K(γGlu-C18-diacid)-OH,
(SEQ ID NO: 19)
HADGTFTSDYSIILD-K(2xAEEA-γGlu-C18-diacid)-
LAARDFINWLIQTKGKKNDWKHNITQ-OH,
(SEQ ID NO: 20)
HADGTFTSDYSTILD-K(2xAEEA-γGlu-C18-diacid)-
LAARDFINWLIQTKGKKNDWKHNITQ-OH,
(SEQ ID NO: 21)
HADGTFTSDYST-Aib-LD-K(2xAEEA-γGlu-C18-diacid)-
LAARDFINWLIQTKGKKNDWKHNITQ-OH,
SEQ ID NO: 22)
HADGTFTSDYSTILDK-K(2xAEEA-γGlu-C18-diacid)-
AARDFINWLIQTKGKKNDWKHNITQ-OH,
(SEQ ID NO: 23)
HADGTFTSDYSTILDKLAARDFINWLIQTKGK-K(2xAEEA-γGlu-C18-diacid)-
NDWKHNITQ-OH,
(SEQ ID NO: 24)
HADGTFTSDYSTILDKLAARDFINWLIQTKGKKNDW-K(2xAEEA-γGlu-C18-
diacid)-HNITQ-OH,
(SEQ ID NO: 25)
HADGTFTSDYSTILDKLAARDFINWLIQTKGKKNDWKHNITQ-K(2xAEEA-γGlu-
C18-diacid)-OH,
SEQ ID NO: 26)
HADGTFTSDYST-Aib-LDKLAARDFINWLIQTKGKKNDWKHNITQ-K(2xAEEA-
γGlu-C18-diacid)-OH,
(SEQ ID NO: 27)
H-Aib-DGTFISDYSTILDALAARDFINWLIQTKIT-K(2xAEEA-γGlu-C18-diacid)-OH,
(SEQ ID NO: 45)
HADGTFISDYSTILDK-(2xAEEA-γGlu-C18-diacid)-LAARDFINWLIQTKGKK-OH,
(SEQ ID NO: 46)
HADGTFISDYST-Aib-LDK-(2xAEEA-γGlu-C18-diacid)-LAARDFINWLIQTKGKK-
OH,
(SEQ ID NO: 47)
HADGTFISDYSTILDNK-(2xAEEA-γGlu-C18-diacid)-AARDFINWLIQTKGKK-OH,
(SEQ ID NO: 48)
HADGTFISDYSTILDNLAARDFINWLIQTKGKK-(2xAEEA-γGlu-C18-diacid)-OH,
(SEQ ID NO: 49)
HADGTFISDYSTILDNLAARDFINWLIQTKGKKK-(2xAEEA-γGlu-C18-diacid)-OH,
or
(SEQ ID NO: 50)
HADGTFISDYST-Aib-LDNLAARDFINWLIQTKGKKK-(2xAEEA-γGlu-C18-diacid)-
OH,

or a functional variant thereof, wherein said functional variant comprises 1 or 2 individual amino acid substitutions.

### Activities of the Peptide Triple Agonists

In one embodiment a functional variant or fragment retains the same biological activity or capabilities as the native peptide or the peptide from which it is derived.

In one embodiment the peptide triple agonist including functional variants or fragments thereof is capable of one or more of:
a. binding to GIPR, GLP-1R and GLP-2R, and/or
b. activation of GIPR, GLP-1R and GLP-2R, and/or
c. stimulation of GIPR-, GLP-1R- and GLP-2R-activation, such as GIPR-, GLP-1R- and GLP-2R-mediated CAMP production, and/or
d. inhibiting bone resorption, and/or
e. stimulating bone formation.

In one embodiment the peptide triple agonist including functional variants or fragments thereof is capable of reducing GIPR internalization compared to native hGIP.

This effect may be desired because it may lead to a sustained pharmacological effect in long term treatment (weeks, months, years) and may not cause desensitization of the GIPR. Desensitization of the GIPR is discussed in Sameer et al. Mol Cell Biol. 2014 Oct. 1; 34(19):3618-29. doi: 10.1128/MCB.00256-14.

In one embodiment the peptide triple agonist including functional variants or fragments thereof is capable of reducing GLP-1R internalization compared to native hGLP-1.

In one embodiment the peptide triple agonist including functional variants or fragments thereof is capable of reducing GLP-2R internalization compared to native hGLP-2.

Peptide triple agonists of the present disclosure may thus be biased towards one receptor, for example:
some peptide triple agonists of the present disclosure may be capable of reducing internalization of only one of GIPR, GLP-1R and GLP-2R compared to native hGIP, hGLP-1, hGLP-2, e.g. they may be capable of reducing internalization of GIPR only, or GLP-1R only, or GLP-2R only and thus be bias for GIPR or GLP-1R or GLP-2R;
some peptide triple agonists of the present disclosure may be capable of reducing internalization of two of GIPR, GLP-1R and GLP-2R, e.g. they may be capable of reducing internalization of GIPR and GLP-1R, but they may not be capable of reducing internalization of GLP-2R and thus be biased for GLP-2R;
some peptide triple agonists of the present disclosure may be capable of reducing internalization of all three GIPR, GLP-1R and GLP-2R; such peptide triple agonists of the present disclosure are thus unbiased.

In one embodiment the peptide triple agonist as well as functional variants or fragments thereof is a full agonist of GIPR, GLP-1R and GLP-2R.

In one embodiment the peptide triple agonists are capable of binding to and activating GIPR. In some embodiments, the GIPR is the human GIPR (Uniprot accession number P48546). In one embodiment the peptide triple agonists are capable of binding to and activating GLP-1R. In some embodiments, the GLP-1R is the human GLP-1R (Uniprot accession number P43220). In one embodiment the peptide triple agonists are capable of binding to and activating GLP-2R. In some embodiments, the GLP-2R is the human GLP-2R (Uniprot accession number 095838).

Activities of the peptide triple agonists of the present disclosure may be assessed experimentally by means of a CAMP assay, as the one described in “Examples” herein. Such a CAMP assay can be used to determine EC₅₀ and Eₘₐₓ, as well as other relevant parameters.

In one embodiment the peptide triple agonists provided herewith are capable of
a. activating the human GIPR with a potency (Eₘₐₓ values) which is at least 65% of the efficacy by which native human GIP activates the human GIPR, such as at least 70%, such as at least 75%, such as at least 80%, such as at least 85%, such as at least 90%, such as at least 95% of the efficacy by which native human GIP activates the human GIPR;
b. activating the human GLP-2R with an efficacy (Eₘₐₓ values) which is at least 65% of the efficacy by which native human GLP-2 activates the human GLP-2R, such as at least 70%, such as at least 75%, such as at least 80%, such as at least 85%, such as at least 90%, such as at least 95% of the efficacy by which native human GLP-2 activates the human GLP-2R, and
a. activating the human GLP-1R with an efficacy (Eₘₐₓ values) which is at least 65% of the efficacy by which native human GLP-1 activates the human GLP-1R, such as at least 70%, such as at least 75%, such as at least 80%, such as at least 85%, such as at least 90%, such as at least 95% of the efficacy by which native human GLP-1 activates the human GLP-1R.

In one embodiment the peptide triple agonists provided herewith are capable of
activating the GIPR with the same or increased potency by which native GIP activates the GIPR;
activating the GLP-1R with the same or increased potency by which native GLP-1 activates the GLP-1R and
activating the GLP-2R with the same or increased potency by which native GLP-2 activates the GLP-2R.

In one embodiment the peptide triple agonists provided herewith
a. have an EC₅₀ towards the human GIPR of 10 nM or less;
b. have an EC₅₀ towards the human GLP-2R of 50 nM or less, and
c. have an EC₅₀ towards the human GLP-1R of 12 nM or less, such as of 10 nm or less, such as of 9 nM or less.

In one embodiment the peptide triple agonists provided herewith
a. have an EC₅₀ towards the human GIPR of 10 nM or less, such as of 9 nM or less; such as of 8 nM or less, such as of 7 nM or less, such as of 6 nM or less, such as of 5 nM or less, such as of 4 nM or less, such as of 3 nM or less, such as of 2 nM or less, such as of 1 nM or less;
b. have an EC₅₀ towards the human GLP-2R of 50 nM or less, such as of 40 nM or less, such as of 30 nM or less, such as of 25 nM or less, such as of 20 nM or less, such as of 15 nM or less, such as of 10 nM or less; and
c. have an EC₅₀ towards the human GLP-1R of 12 nM or less, such as of 10 nm or less, such as of 9 nM or less such as of 8 nM or less, such as of 7 nM or less, such as of 6 nM or less, such as of 5 nM or less, such as of 4 nM or less, such as of 3 nM or less

### Medicament/Medical Use

It is an aspect of the present disclosure to provide a peptide triple agonist, a nucleic acid construct encoding a peptide triple agonist, a delivery vehicle comprising a nucleic acid construct encoding a peptide triple agonist, as well a composition comprising the peptide triple agonist, as defined herein elsewhere, for use as a medicament.

It is thus an aspect of the present disclosure to provide a peptide triple agonist as defined herein for use in a method of inhibiting bone resorption and/or stimulating bone formation.

It is also an aspect of the present disclosure to provide a peptide triple agonist as defined herein for use in a method of inhibiting bone resorption and/or stimulating bone formation.

Also provided is the use of a peptide triple agonist as defined herein for the manufacture of a medicament for inhibiting bone resorption and/or stimulating bone formation.

Also provided is a method of inhibiting bone resorption and/or stimulating bone formation, said method comprising administering a therapeutically effective amount of a peptide triple agonist as defined herein to an individual in need thereof.

It is a further aspect of the present disclosure to provide a peptide triple agonist as defined herein for use in a method of treating a bone disorder. In one embodiment the bone disorder is a disorder associated with increased bone resorption and/or reduced bone formation. In one embodiment the bone disorder is associated with poor or reduced bone density.

Also provided is a peptide triple agonist as defined herein for use in a method of treating a bone disorder, including treating, preventing and alleviating said bone disorder.

Also disclosed is a peptide triple agonist disclosed herein for use in a method of treating a bone disorder.

Bone density or bone mineral density (BMD) is the amount of bone mineral in bone tissue. The concept is of mass of mineral per volume of bone (relating to density in the physics sense), although clinically it is measured by proxy according to optical density per square centimetre of bone surface upon imaging. Bone density measurement is used in clinical medicine as an indirect indicator of osteoporosis/osteopenia and fracture risk. It is measured by a procedure called densitometry. There is a statistical association between poor bone density and higher probability of fracture. Bone density measurements are used to screen people for osteoporosis risk and to identify those who might benefit from measures to improve bone strength.

The T-score is the relevant measure when screening for osteoporosis. It is the bone mineral density (BMD) at the site when compared to the young normal reference mean. The criteria of the World Health Organization are:
Normal is a T-score of −1.0 or higher
Osteopenia is defined as between −1.0 and −2.5
Osteoporosis is defined as −2.5 or lower, meaning a bone density that is two and a half standard deviations below the mean of a young normal reference.

In one embodiment the bone disorder is associated with a T-score of −1.0 or lower, such as between −1.0 and −2.5, such as −2.5 or lower.

In one embodiment there is provided the use of a peptide triple agonist as defined herein for the manufacture of a medicament for treating a bone disorder.

Also provided is a method of treating a bone disorder, said method comprising administering a therapeutically effective amount of a peptide triple agonist as defined herein to an individual in need thereof.

An individual in need as referred to herein, is an individual that may benefit from the administration of a triple agonist peptide. Such an individual may suffer from a bone disorder or be in risk of suffering therefrom. The individual may be any human being, male or female, infant, middle-aged or old. The disorder to be treated or prevented in the individual may relate to the age of the individual, the general health of the individual, the medications used for treating the individual and whether or not the individual has a prior history of suffering from diseases or disorders that may have or have induced a bone density disorder.

In one embodiment the bone disorder is selected from the group consisting of osteopenia, osteoporosis, severe osteoporosis, post-menopausal osteoporosis, iodiopathic osteoporosis, osteomalacia, rickets, osteitis fibrosa cystica (OFC) and Paget's disease of bone.

In one embodiment the bone disorder is osteopenia.

In one embodiment the bone disorder is osteoporosis.

In one embodiment the bone disorder is post-menopausal osteoporosis.

In one embodiment the bone disorder is iodiopathic osteoporosis, such as juvenile iodopathic osteoporosis.

It is a further aspect of the present disclosure to provide a peptide triple agonist as defined herein for use in a method of treating osteoporosis, such as idiopathic osteoporosis, in an individual suffering from Duchenne muscular dystrophy (DMD) or cerebral Palsy.

In one embodiment the bone disorder is a secondary disorder, such as a secondary disorder in an individual suffering from Duchenne muscular dystrophy (DMD) or cerebral Palsy.

In one embodiment the bone disorder is a secondary disorder, such as a secondary disorder in an individual who is undergoing steroid, such as glucocorticoid treatment and/or is subject to immobility.

Idiopathic osteoporosis refers to the development of osteopenia and fractures with minimal or no trauma in otherwise young, healthy individuals who are not postmenopausal or have other, identifiable secondary causes of osteoporosis.

Idiopathic osteoporosis refers to the development of osteopenia and fractures with minimal or no trauma in otherwise young, healthy individuals who are not postmenopausal or have other, identifiable secondary causes of osteoporosis.

Also disclosed is a peptide triple agonist selected from the group consisting of SEQ ID NO: s XXX, or a functional variant thereof, or a functional fragment thereof, for use in a method of treating a bone disorder selected from the group consisting of osteopenia, osteoporosis, severe osteoporosis, osteomalacia, rickets, osteitis fibrosa cystica (OFC) and Paget's disease of bone.

In one embodiment there is provided a peptide triple agonist selected from the group consisting of SEQ ID NO:s 42 to 44, or a functional variant thereof, or a functional fragment thereof, for use in a method of treating osteopenia.

In one embodiment there is provided a peptide triple agonist selected from the group consisting of SEQ ID NO:s 42 to 44, or a functional variant thereof, or a functional fragment thereof, for use in a method of treating osteoporosis.

In one embodiment there is provided a peptide triple agonist selected from the group consisting of SEQ ID NO:s 42 to 44, or a functional variant thereof, or a functional fragment thereof, for use in a method of treating osteoporosis in an individual suffering from Duchenne muscular dystrophy (DMD) or cerebral Palsy.

### Combination Therapy

It is also an aspect to provide a peptide triple agonist as defined herein for use in combination with a further active pharmaceutical ingredient. Said further active ingredient is in one embodiment useful for treating a bone disorder, such as a bone disorder associated with reduced bone density.

In one embodiment the further active pharmaceutical ingredient is selected from the group consisting of Bisphosphonates including Alendronate (Fosamax), Risedronate (Actonel, Atelvia, Benet), Ibandronate (Boniva), Zoledronic acid (Reclast, Aclasta, Zometa), Etidronic acid (Didronel), Pamidronic acid (Aredia/Pamimed), Tiludronic acid (Skelid); estrogen replacement therapy; hormone therapies; hormone-like medications including raloxifene (Evista); Calcitonin (Fortical and Miacalcin), Denosumab (Prolia); Teriparatide (Forteo); Vitamin D (alfacalcidol or calcitriol); calcium or phosphorus supplement.

In one embodiment the further active pharmaceutical ingredient increases the half-life of the present peptide triple agonist. In one embodiment the further active pharmaceutical ingredient is an inhibitor of dipeptidyl peptidase 4 (DPP-4/DDP-IV inhibitors), or gliptins. Examples include Sitagliptin, Vildagliptin, Saxagliptin, Linagliptin, Gemigliptin, Anagliptin, Teneligliptin, Alogliptin, Trelagliptin, Dutogliptin, and Omarigliptin (MK-3102).

### Nucleic Acid Construct Encoding Triple Agonist Peptide

In one embodiment there is provided a nucleic acid construct encoding a triple agonist peptide as defined herein. In one embodiment said nucleic acid construct will be able to continuously express said peptide for a prolonged period of time; such as at least 1 month, for example at least 2 months, such as at least 3 months, for example at least 4 months, such as at least 5 months, for example at least 6 months, such as at least 7 months, for example at least 8 months, such as at least 9 months, for example at least 12 months.

It is thus an aspect to provide a nucleic acid construct encoding a peptide triple agonist as defined herewith.

In one embodiment there is provided a nucleic acid construct encoding a peptide triple agonist of SEQ ID NO: 42 to 44, or a functional variant thereof, or a functional fragment thereof, or any peptide triple agonist disclosed herein.

It is also an aspect to provide a nucleic acid construct encoding a triple agonist peptide as defined herein for use in a method of treating a bone disorder.

By nucleic acid construct is understood a genetically engineered nucleic acid. The nucleic acid construct may be a non-replicating and linear nucleic acid, a circular expression vector or an autonomously replicating plasmid. A nucleic acid construct may comprise several elements such as, but not limited to genes or fragments of same, promoters, enhancers, terminators, poly-A tails, linkers, polylinkers, operative linkers, multiple cloning sites (MCS), markers, STOP codons, internal ribosomal entry sites (IRES) and host homologous sequences for integration or other defined elements. It is to be understood that the nucleic acid construct according to the present invention may comprise all or a subset of any combination of the above-mentioned elements.

Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Further, nucleic acid constructs according to the present invention may be synthesized without template, and may be obtained from various commercial suppliers (e.g. Genscript Corporation).

In one embodiment, the nucleic acid constructs are naked DNA constructs comprising sequences encoding the triple agonist peptide.

### Delivery Vehicles

It is also an aspect to provide the nucleic acid construct as described herein above comprised within a delivery vehicle. A delivery vehicle is an entity whereby a nucleotide sequence or polypeptide or both can be transported from at least one media to another. Delivery vehicles are generally used for expression of the sequences encoded within the nucleic acid construct and/or for the intracellular delivery of the construct or the polypeptide encoded therein.

In one embodiment, there is provided a delivery vehicle comprising the nucleic acid construct as defined herein. A delivery vehicle may be selected from the group consisting of: RNA based vehicles, DNA based vehicles/vectors, lipid based vehicles (such as a liposome), polymer based vehicles (such as a cationic polymer DNA carrier), colloidal gold particles (coating) and virally derived DNA or RNA vehicles or vectors.

Methods of non-viral delivery include physical (carrier-free delivery) and chemical approaches (synthetic vector-based delivery).

Physical approaches, including needle injection, gene gun, jet injection, electroporation, ultrasound, and hydrodynamic delivery, employ a physical force that permeates the cell membrane and facilitates intracellular gene transfer. Said physical force may be electrical or mechanical.

Examples of chemical delivery vehicles include, but are not limited to: biodegradable polymer microspheres, lipid based formulations such as liposome carriers, cationically charged molecules such as liposomes, calcium salts or dendrimers, lipopolysaccharides, polypeptides and polysaccharides.

Another embodiment comprises a vector which herein is denoted a viral vector (i.e. not a virus) as a delivery vehicle. Viral vectors according to the present invention are made from a modified viral genome, i.e. the actual DNA or RNA forming the viral genome, and introduced in naked form. Thus, any coat structures surrounding the viral genome made from viral or non-viral proteins are not part of the viral vector.

The virus from which the viral vector is derived may be selected from the non-exhaustive group of: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

In one embodiment, said viral vectors may be selected from the group consisting of adenoviruses, lentiviruses, adeno-associated viruses (AAV) and recombinant adeno-associated viruses (rAAV). In one preferred embodiment, said viral vector is a therapeutic rAAV vector such as a therapeutic rAAV vector.

An adenovirus is a group of double-stranded DNA containing viruses. Adenoviruses can be genetically modified making them replication incompetent or conditionally replication incompetent. In this form, as adenoviral constructs or adenovectors, they can be used as gene delivery vehicles for vaccination or gene therapy.

### Recombinant Cell

Another aspect of relates to a cell comprising the nucleic acid construct as defined herein. Such a recombinant cell can be used a tool for in vitro research, as a delivery vehicle for the nucleic acid construct or as part of a gene-therapy regime. The nucleic acid construct can be introduced into cells by techniques well known in the art which include microinjection of DNA into the nucleus of a cell, transfection, electroporation, lipofection/liposome fusion and particle bombardment. Suitable cells include autologous and non-autologous cells, and may include xenogenic cells.

### Method of Preparation (Peptide)

The triple agonist peptides as defined herein may be prepared by any methods known in the art; such as by standard peptide-preparation techniques including solution synthesis or Merrifield-type solid phase synthesis.

In one embodiment a peptide according to the invention is synthetically made or produced. The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

In one embodiment the peptide or peptide sequences of the invention are produced synthetically, in particular, by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

Peptides may be synthesised either batch-wise on a fully automated peptide synthesiser using 9-fluorenylmethyloxycarbonyl (Fmoc) or tert-Butyloxycarbonyl (Boc) as N-a-amino protecting group and suitable common protection groups for side-chain functionalities.

After purification such as by reversed phase HPLC, peptides may be further processed to obtain for example C- or N-terminal modified isoforms. The methods for terminal modification are well-known in the art.

For example, the following methods for conjugation of fatty acid can be used. After the last N-terminal N-Fmoc is removed, the N-terminal amino group is acylated by the treatment of 3 equiv 1,16-Hexadecanedioic acid in the presence of 3 equiv HATU and 6 equiv DIPEA. As an alternative, Lysine with Dde protected side chain is used for the solid phase peptide synthesis in the acylation position. After the peptide chain is assembled, the N-terminal amino group is capped by 3 equiv Boc20 in the presence of 6 equiv DIPEA. Then the protection group Dde is removed by the treatment of 2% hydrazine/DMF (v/v). As an alternative, 3 equiv palmitoyl chloride can be used for palmitoylation in the presence of 6 equiv DIPEA. Or 3 equiv 1,16-Hexadecanedioic acid can be activated by HATU/DIPEA and then conjugated to the side chain of Lysine. As a further alternative, Fmoc-γ-Glu(tBu)-OH or Fmoc-4-Abu-OH or Fmoc-B-Ala-OH or Fmoc-AEEAc-OH are conjugated to the side chain of Lys using a standard protocol of DIC/HOBT. The N-terminal amino groups of unnatural amino acids is then palmitoylated by the treatment of 3 equiv palmitoyl chloride in the presence of 6 equiv DIPEA.

### Pharmaceutical Composition and Formulation

Whilst it is possible for the triple agonist peptide as defined herewith to be administered as the raw chemical (peptide), it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, also provided is a pharmaceutical formulation, which comprises a triple agonist peptide as defined herein, or a pharmaceutically acceptable salt or ester thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant peptide compounds, where they can be prepared, are also intended to be covered. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The peptide compounds may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutically acceptable salt of the peptide of the invention is preferably in solution with a physiologically acceptable pH, i.e. the solution comprising the peptide salt preferably has a pH acceptable for clinical use.

Provided herewith is a composition comprising a peptide triple agonist as defined herein; or a multimeric compound comprising said peptide triple agonist; or a nucleic acid construct comprising/encoding said peptide triple agonist.

### Administration and Dosage

In one embodiment of the present disclosure, a peptide or a nucleic acid construct encoding said peptide, or a composition comprising a peptide as defined herein is administered to individuals in need of treatment in pharmaceutically effective doses or a therapeutically effective amount. The dosage requirements will vary with the particular drug composition employed, the route of administration and the particular subject being treated, which depend on the severity and the sort of the disorder as well as on the weight and general state of the subject. It will also be recognized by one skilled in the art that the optimal quantity and spacing of individual dosages will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

In one embodiment the peptide or composition is administered at least once daily, such as once daily, such as twice daily, such as thrice daily, such as four times daily, such as five times daily.

A dose may also be administered in intermittent intervals, or intervals, whereby a dose is not administered every day. Rather one or more doses may be administered every second day, every third day, every fourth day, every fifth day, every sixth day, every week, every second week, every third week, every fourth week, every fifth week, every sixth week, or intervals within those ranges (such as every 2 to 4 weeks, or 4 to 6 weeks).

In one embodiment the peptide or composition is administered once weekly. Constant exposure to the effects of the peptide triple agonist of the present disclosure may be obtained with such an administration profile.

The skilled person knows that if the number of daily administrations is increased, the dose to be administered in each administration may be decreased accordingly. Likewise, if the duration of each administration is decreased, the dosage may be increased accordingly.

### Routes of Administration

It will be appreciated that the preferred route of administration will depend on the general condition and age of the subject to be treated, the nature of the condition to be treated, the location of the tissue to be treated in the body and the active ingredient chosen.

### Systemic Treatment

For systemic treatment the route of administration is capable of introducing the peptide, nucleic acid construct encoding said peptide, or the composition comprising the peptide into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, compound be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agent may be administered topically to cross the skin.

### Local Treatment

The peptide, or a nucleic acid construct encoding said peptide, or a composition comprising a peptide as defined herein may in one embodiment be used as a local treatment, i.e. be introduced directly to the site(s) of action. Accordingly, it may be applied to the skin or mucosa directly, or it may be injected into the site of action, for example into the diseased tissue or to an end artery leading directly to the diseased tissue.

### Kit-of-Parts

The present invention also relates to a kit-of-parts comprising one or more of the agents described above (a peptide, a nucleic acid construct or a composition), and at least one additional or further component.

A kit of parts in one embodiment comprises one or more of the agents as defined herein for treatment, prevention or alleviation of a bone disorder osteoporosis and osteopenia. Kits as defined herein allows for simultaneous, sequential or separate administration of the active agent according to the present invention and/or one or more second active ingredients as described herein elsewhere.

### EXAMPLES

### Example 1. Peptides Triple Agonists of GIPR, GLP-2R and GLP-1R

### Materials and Methods

### Materials

All applied peptides were synthesized by WuXi AppTec, Shanghai, China, with a purity of at least 95% or above, determined by HPLC analysis, and the correct molecular weight was checked using mass spectrometry. The human GIPR, GLP-2R and GLP-1R applied in the in vitro experiments can be purchased by Origene, Rockville, USA. Human N-terminally SNAP-tagged GIPR was custom produced by Cisbio, Codolet, France. C10, C12 or C16 or C16-diacid acyl groups were added on position 16, 17, 20, 30, 33, 37 or C-terminal amino acid residue (C-term) as provided in Table 1 to improve the potency of the triple agonists.

### Results:

The peptide triple agonists listed in Table 1 were obtained.

| Comp. | SEQ | | Lip. | Fatty | |
|---|---|---|---|---|---|
| No. | ID NO: | Length | Pos. | acid | Linker |
| 1 | 1 | 33 | C-term | C10 | |
| 2 | 2 | 33 | C-term | C10 | |
| 3 | 3 | 33 | C-term | C10 | |
| 4 | 4 | 33 | C-term | C10 | |
| 5 | 5 | 33 | C-term | C10 | |
| 6 | 6 | 33 | C-term | C10 | |
| 7 | 7 | 33 | C-term | C10 | |
| 8 | 8 | 33 | C-term | C10 | |
| 9 | 9 | 33 | C-term | C10 | |
| 10 | 10 | 33 | C-term | C10 | |
| 11 | 11 | 33 | C-term | C10 | |
| 12 | 12 | 33 | C-term | C10 | |
| 13 | 13 | 33 | C-term | C10 | |
| 14 | 14 | 33 | C-term | C10 | |
| 15 | 15 | 33 | C-term | C10 | |
| 16 | 16 | 33 | C-term | C10 | |
| 17 | 1 | 33 | C-term | C10 | yGlu |
| 18 | 17 | 33 | 20 | C18-diacid | 2xAEEA-yGlu |
| 19 | 18 | 33 | C-term | C18-diacid | yGlu |
| 20 | 19 | 42 | 16 | C18-diacid | 2xAEEA-yGlu |
| 21 | 20 | 42 | 16 | C18-diacid | 2xAEEA-yGlu |
| 22 | 21 | 42 | 16 | C18-diacid | 2xAEEA-yGlu |
| 23 | 22 | 42 | 17 | C18-diacid | 2xAEEA-yGlu |
| 24 | 23 | 42 | 33 | C18-diacid | 2xAEEA-yGlu |
| 25 | 24 | 42 | 37 | C18-diacid | 2xAEEA-yGlu |
| 26 | 25 | 43 | C-term | C18-diacid | 2xAEEA-yGlu |
| 27 | 26 | 43 | C-term | C18-diacid | 2xAEEA-yGlu |
| 28 | 27 | 33 | C-term | C18-diacid | 2xAEEA-yGlu |
| 29 | 28 | 33 | 11 | C16 | yGlu |
| 30 | 45 | 33 | 16 | C18-diacid | 2xAEEA-yGlu |
| 31 | 46 | 33 | 16 | C18-diacid | 2xAEEA-yGlu |
| 32 | 47 | 33 | 17 | C18-diacid | 2xAEEA-yGlu |
| 33 | 48 | 33 | C-term | C18-diacid | 2xAEEA-yGlu |
| 34 | 49 | 34 | C-term | C18-diacid | 2xAEEA-yGlu |
| 35 | 50 | 34 | C-term | C18-diacid | 2xAEEA-yGlu |

### Example 2. Potency

### Methods

COS-7 cells were cultured at 10% CO₂ and 37° C. in Dulbecco's Modified Eagle Medium (DMEM) 1885 supplemented with 10% fetal bovine serum (FBS), 2 mM glutamine, 180 units/ml penicillin, and 45 g/ml streptomycin. Transient transfection of COS-7 cells was performed using the calcium phosphate precipitation method as previously described (van der Velden et al., 2021) prior to experiment.

Transiently transfected COS-7 cells expressing either human GIPR, GLP-2R or GLP-1R were seeded in white 96-well plates the day after transfection with a density of 35.000 cells/well. The next day the assay was initiated by washing with HEPES buffered saline (HBS) and followed by an incubation step with assay buffer (HBS added 1 mM isobutyl-1-methylxanthine (IBMX)) for 30 min at 37° C. The ligands were then added and incubated for an additional 30 min at 37° C. After ligand incubation, the HitHunter® CAMP assay (Eurofins DiscoverX, Fremont, 150 USA) was carried out according to the manufacturer's instructions. Luminescence was measured by PerkinElmer™ EnVision 2014 Multilabel Reader (PerkinElmer, Waltham, MA). The potencies (EC₅₀ values) were determined by nonlinear regression using GraphPad Prism 9 (San Diego, California, USA). Sigmoid curves were fitted logistically. The efficacies (Eₘₐₓ values) were determined by normalization to the highest concentration applied of the endogenous ligand for each receptor.

### Results

From table 2, 3 and 4 it can be seen that by attaching a fatty acid molecule at a position in the C-terminal region, for example position 16, 17, 20, 33, 37 and at the most C-terminal amino acid, potent GIPR/GLP-2R/GLP-1R triple agonists can be obtained. See for comparison comp. no. 29 that lipidation in e.g. position 11 of the N-terminal region leads to little or no GLP-1R activity.

Attachment of a fatty acid at the C-terminus of compounds with 1-33 or 1-34 backbone leads to reduction in GLP-2R activation (see e.g. Compound 19). The same loss in activation is not observed for compounds with a fatty acid attached to the C-terminus at position 42 or 43 of compounds with a longer backbone (1-43 or 1-44), see e.g. Compounds 24-26.

Substitution of Aspartic acid (D) in position 3 with a Glutamic acid (E) appears to reduce potency towards all three receptors GIPR, GLP-2R and GLP-1R and thus gives less potent GIPR/GLP-2R/GLP-1R triple agonists, see e.g. Compound 11. Similarly, substitution of Isoleucine (I) in position 13 with a Glutamic acid (E) appears to reduce potency towards all three receptors GIPR, GLP-2R and GLP-1R and thus gives less potent GIPR/GLP-2R/GLP-1R triple agonists, see e.g. Compound 13.

Substitution of Histidine (H) in position 1 with a Tyrosine (Y) appears to increase activation of GIPR while at the same time it seems to reduce GLP-1R activation, see e.g. Compound 16.

Threonine in position 7 appears to be highly acceptable by all three receptors GIPR, GLP-2R and GLP-1R and thus gives potent GIPR/GLP-2R/GLP-1R triple agonists, in particular increasing GLP-1R activity.

Serine(S) in position 11 appears to increase potency towards GLP-2R and thus gives potent GIPR/GLP-2R/GLP-1R triple agonists biased for GLP-2R.

Attaching a fatty acid in position 16 appears to be particular suitable for obtaining GIPR/GLP-2R/GLP-1R triple agonists with high potency at all three receptors.

Attaching a fatty acid in position 17, see e.g. Compound 32, compared to attachment of a fatty acid in position 16, see e.g. Compound 30, seems to cause a loss in GIP potency in cAMP (>10 fold) as well as a small loss in GLP-2R activation, while no loss towards GLP-1R activation.

Alanine in position 16 appears to be very suitable for obtaining GIPR/GLP-2R/GLP-1R triple agonists, in particular increasing GLP-1R activity.

A peptide comprising the sequence NDWKHNITQK (SEQ ID NO: Z) (C-terminal part of hGIP (34-42)) appears to be very suitable for obtaining potent GIPR/GLP-2R/GLP-1R triple agonists.

C-terminal carboxylation (—COOH) seems to increase GIPR and GLP-1R activation and decrease GLP-2R activation, see e.g. Compound 2.

TABLE 2
EC₅₀ (potency) and Emax (Efficacy) of the tested peptide triple
agonists of GIPR, GLP-2R and GLP-1R with respect to human GIPR.

| Comp. | pEC₅₀ | | EC₅₀ (nM) | Efficacy | | |
|---|---|---|---|---|---|---|
| No. | GIPR | SD | GIPR | GIPR | SD | n= |
| 1 | 9.6 | 0.1 | 0.251 | 93 | 7 | 2 |
| 2 | 9.6 | 0.1 | 0.282 | 99 | 1 | 2 |
| 3 | 9.2 | 0.3 | 0.631 | 80 | 2 | 2 |
| 4 | 9.0 | 0.2 | 1.122 | 102 | 2 | 2 |
| 5 | 9.8 | 0.1 | 0.158 | 111 | 13 | 2 |
| 6 | 9.2 | 0.4 | 0.584 | 97 | 4 | 3 |
| 7 | 9.1 | 0.3 | 0.794 | 96 | 22 | 2 |
| 8 | 9.0 | 0.4 | 1.122 | 99 | 4 | 2 |
| 9 | 9.2 | 0.1 | 0.708 | 104 | 3 | 2 |
| 10 | 9.3 | 0.0 | 0.501 | 127 | 12 | 2 |
| 11 | 7.9 | 0.3 | 13.594 | 89 | 30 | 3 |
| 12 | 8.8 | 0.4 | 1.468 | 107 | 20 | 3 |
| 13 | 8.1 | 0.3 | 7.943 | 117 | 16 | 2 |
| 14 | 8.9 | 0.4 | 1.259 | 91 | 7 | 2 |
| 15 | 9.1 | 0.6 | 0.794 | 98 | 12 | 3 |
| 16 | 9.0 | 0.4 | 1.000 | 99 | 1 | 2 |
| 17 | 9.3 | 0.6 | 0.479 | 97 | 17 | 5 |
| 18 | 8.3 | 0.1 | 5.012 | 82 | 18 | 2 |
| 19 | 9.2 | 0.0 | 0.631 | 92 | 14 | 2 |
| 20 | 9.9 | 0.1 | 0.126 | 112 | 30 | 3 |
| 21 | 9.4 | 0.1 | 0.398 | 104 | 21 | 2 |
| 22 | 9.0 | 0.1 | 1.122 | 93 | 23 | 2 |
| 23 | 8.2 | 0.2 | 7.079 | 82 | 3 | 2 |
| 24 | 9.4 | 0.3 | 0.398 | 96 | 25 | 2 |
| 25 | 9.4 | 0.3 | 0.398 | 107 | 16 | 2 |
| 26 | 9.5 | 0.1 | 0.316 | 106 | 16 | 2 |
| 27 | 9.3 | 0.1 | 0.562 | 124 | 30 | 2 |
| 28 | 10.2 | 0.2 | 0.071 | 119 | 18 | 2 |
| 29 | 9.2 | 0.1 | 0.681 | 115 | 8 | 3 |
| 30 | 10.3 | 0.2 | 0.056 | 98 | 7.2 | 2 |
| 31 | 9.9 | 0.2 | 0.115 | 85 | 8.7 | 2 |
| 32 | 8.8 | 0.1 | 1.698 | 80 | 8.5 | 2 |
| 33 | 9.9 | 0.1 | 0.141 | 88 | 6.4 | 2 |
| 34 | 9.8 | 0.2 | 0.158 | 103 | 15.6 | 2 |
| 35 | 9.5 | 0.1 | 0.288 | 99 | 36.1 | 2 |

TABLE 3
EC₅₀ (potency) and Emax (Efficacy) of the tested peptide triple
agonists of GIPR, GLP-2R and GLP-1R with respect to human GLP-2R.

| Comp. | logEC₅₀ | | EC₅₀ (nM) | Efficacy | | |
|---|---|---|---|---|---|---|
| No. | GLP-2R | SD | GLP-2R | GLP-2R | SD | n= |
| 1 | 8.7 | 0.3 | 2.0 | 96 | 22 | 3 |
| 2 | 8.0 | 0.4 | 11.2 | 105 | 13 | 2 |
| 3 | 8.6 | 0.5 | 2.8 | 101 | 4 | 2 |
| 4 | 7.6 | 0.4 | 28.2 | 92 | 8 | 2 |
| 5 | 8.7 | 0.1 | 2.0 | 110 | 18 | 2 |
| 6 | 8.0 | 0.3 | 9.3 | 99 | 14 | 3 |
| 7 | 7.9 | 0.3 | 12.6 | 97 | 1 | 2 |
| 8 | 8.2 | 0.2 | 7.1 | 94 | 5 | 2 |
| 9 | 8.0 | 0.2 | 11.2 | 108 | 12 | 2 |
| 10 | 8.0 | 0.5 | 11.2 | 103 | 39 | 2 |
| 11 | 7.6 | 0.5 | 25.1 | 68 | 19 | 3 |
| 12 | 8.2 | 0.3 | 6.8 | 77 | 6 | 3 |
| 13 | 7.6 | 0.1 | 25.1 | 86 | 6 | 2 |
| 14 | 7.8 | 0.3 | 15.8 | 92 | 13 | 2 |
| 15 | 8.2 | 0.3 | 7.1 | 90 | 12 | 4 |
| 16 | 8.1 | 0.4 | 8.9 | 93 | 0 | 2 |
| 17 | 8.3 | 0.3 | 5.5 | 92 | 11 | 5 |
| 18 | 8.8 | 0.1 | 1.6 | 91 | 18 | 2 |
| 19 | 7.5 | 0.0 | 31.6 | 94 | 9 | 2 |
| 20 | 9.5 | 0.2 | 0.3 | 100 | 19 | 3 |
| 21 | 9.2 | 0.3 | 0.6 | 104 | 9 | 2 |
| 22 | 9.3 | 0.2 | 0.6 | 93 | 3 | 2 |
| 23 | 8.5 | 0.2 | 3.5 | 95 | 8 | 2 |
| 24 | 7.7 | 0.4 | 22.4 | 91 | 11 | 2 |
| 25 | 8.1 | 0.4 | 7.9 | 117 | 11 | 2 |
| 26 | 8.3 | 0.4 | 5.6 | 112 | 17 | 2 |
| 27 | 8.5 | 0.1 | 3.2 | 85 | 3 | 2 |
| 28 | 8.7 | 0.1 | 2.2 | 89 | 15 | 2 |
| 29 | 8.7 | 0.2 | 2.2 | 87 | 6 | 3 |
| 30 | 8.8 | 0.1 | 1.4 | 73 | 13.0 | 3 |
| 31 | 9.0 | 0.1 | 1.0 | 79 | 8.1 | 3 |
| 32 | 8.5 | 0.1 | 3.2 | 77 | 0.0 | 2 |
| 33 | 7.2 | 0.1 | 66.1 | 90 | 9.2 | 2 |
| 34 | 7.5 | 0.4 | 29.5 | 91 | 31.8 | 2 |
| 35 | 7.7 | 0.1 | 20.4 | 93 | 12.0 | 2 |

TABLE 4
EC₅₀ (potency) and Emax (Efficacy) of the tested peptide triple
agonists of GIPR, GLP-2R and GLP-1R with respect to human GLP-1R.

| Comp. | logEC₅₀ | | EC₅₀ (nM) | Efficacy | | |
|---|---|---|---|---|---|---|
| No. | GLP-1R | SD | GLP-1R | GLP-1R | SD | n= |
| 1 | 8.3 | 0.4 | 5.01 | 101 | 12 | 3 |
| 2 | 8.6 | 0.4 | 2.82 | 84 | 28 | 2 |
| 3 | 8.8 | 0.1 | 1.78 | 94 | 13 | 2 |
| 4 | 7.6 | 0.3 | 25.12 | 82 | 28 | 2 |
| 5 | 8.8 | 0.5 | 1.78 | 105 | 15 | 2 |
| 6 | 8.2 | 0.5 | 6.81 | 96 | 33 | 3 |
| 7 | 8.6 | 0.4 | 2.82 | 95 | 10 | 2 |
| 8 | 8.7 | 0.4 | 2.24 | 98 | 9 | 2 |
| 9 | 8.3 | 0.4 | 5.62 | 105 | 14 | 2 |
| 10 | 8.4 | 0.1 | 3.98 | 99 | 10 | 2 |
| 11 | 7.4 | 0.3 | 39.81 | 76 | 9 | 3 |
| 12 | 8.0 | 0.2 | 10.00 | 76 | 31 | 3 |
| 13 | 7.6 | 0.3 | 25.12 | 85 | 28 | 2 |
| 14 | 8.1 | 0.4 | 8.91 | 96 | 6 | 2 |
| 15 | 8.3 | 0.3 | 4.73 | 90 | 4 | 4 |
| 16 | 7.3 | 0.2 | 56.23 | 59 | 3 | 2 |
| 17 | 8.2 | 0.1 | 6.68 | 107 | 23 | 5 |
| 18 | 10.2 | 0.1 | 0.06 | 112 | 30 | 2 |
| 19 | 10.2 | 0.1 | 0.06 | 106 | 5 | 2 |
| 20 | 10.4 | 0.2 | 0.04 | 112 | 22 | 3 |
| 21 | 10.3 | 0.1 | 0.05 | 122 | 11 | 2 |
| 22 | 10.5 | 0.5 | 0.04 | 116 | 7 | 2 |
| 23 | 10.3 | 0.3 | 0.05 | 105 | 26 | 2 |
| 24 | 10.3 | 0.3 | 0.05 | 113 | 14 | 2 |
| 25 | 10.3 | 0.1 | 0.05 | 116 | 13 | 2 |
| 26 | 10.4 | 0.3 | 0.04 | 103 | 16 | 2 |
| 27 | 10.3 | 0.4 | 0.05 | 115 | 1 | 2 |
| 28 | 8.4 | 0.1 | 3.98 | 102 | 2 | 2 |
| 29 | 7.1 | 0.4 | 89.13 | 11 | 1 | 2 |
| 30 | 9.3 | 0.2 | 0.5 | 93 | 16.0 | 3 |
| 31 | 9.1 | 0.3 | 0.8 | 89 | 11.5 | 3 |
| 32 | 9.3 | 0.1 | 0.6 | 84 | 9.2 | 2 |
| 33 | 8.4 | 0.1 | 3.6 | 99 | 19.1 | 2 |
| 34 | 8.4 | 0.0 | 3.7 | 97 | 5.7 | 2 |
| 35 | 8.2 | 0.1 | 5.8 | 92 | 1.4 | 2 |

### Example 3: Internalization

### Methods

HEK293A cells were cultured at 5% CO₂ and 37° C. in DMEM-GlutaMAX™-I supplemented with 10% FBS, 180 units/mL penicillin and 45 g/mL streptomycin. These cells were transfected using Lipofectamine 2000 from Thermo Fischer Scientific, Massachusetts, USA according to the manufacturer's instructions for the receptor internalization assay.

HEK293A cells transiently expressing human SNAP-tagged GIPR, human SNAP-tagged GLP-1R or human SNP-tagged GLP-2R were seeded in white 384-well plates on the day of transfection at a density of 15.000 cells/well. The following day the media was removed and new, fresh culture media was added. On day three, the assay was run. First, the cells were labeled with 100 nM Tag-Lite SNAP-Lumi4-Tb (donor) in OptiMEM for 60 min at 37° C. Next, the cells were washed four times with internalization buffer (HBBS supplemented with 1 mM CaCl₂), 1 mM MgCl2 and 20 mM HEPES) followed by addition of 100 UM preheated fluorescein-O′-acetic acid (acceptor). The plate was placed in a 37° C. incubator for 5 minutes prior to ligand stimulation to adjust the temperature before reading the plate. Then the cells were stimulated with 37° C. preheated ligand solution and internalization was measured every third minute for 60 minutes at 37° C. in PerkinElmer™ EnVision 2014 Multilabel Reader (PerkinElmer, Waltham, MA). The AUC values for the sigmoid curves up to the concentration of 1 μM were likewise calculated.

### Results

In general, the data show that the tested triple agonists induced less GIPR internalization than native GIP, and less GLP-2R internalization than native GLP-2, although to different extents. Some of the tested triple agonists also induce less GLP-1R internalization than native GLP-1, while other tested triple agonists induce slightly more internalization than native GLP-1.

Reduced internalization normally correlates with reduced recruitment of beta-arrestin 2.

Attachment of a fatty acid to the C-terminal at position 33 of a triple agonist peptide of the present disclosure correlated with low internalization in general and an internalization bias towards GIPR receptor, with very low or no internalization of GLP-1R and GLP-2R. See for example Compounds no. 1-17, 19, 28, 33-35 (some data reported in Tables 5-7). In contrast, Compounds no. 26 and 27, which are lipidated on the C-terminus at position 43, caused internalization of the GIPR to a higher extent.

The data also show that lipidation in position 16 of compounds having a 1-42 or 1-43 backbone, e.g. Compounds 20, 21, 22, resulted in unbiased internalization on GLP-1 and 2 receptors (comparable to native GLP-1 and GLP-2), but biased internalization on GIPR (reduced internalization compared to native GIP).

The data also show that lipidation in position 16 of compounds having a 1-33, 1-34 backbone, see e.g. Compounds 30, 31, 32, also resulted in biased internalization on GLP-1R, but less biased on GLP-2R. Without being bound to theory, i) a short peptide backbone creates bias towards one of GIPR relative to the endogenous agonist GIP, GLP-1R and/or GLP-2R to a larger extent than a long peptide backbone, and ii)C-terminus lipidation was related to most bias (in terms of lost internalization).

Presence of Aib in position 13 results in slightly larger impairment in internalization (more bias) for the GIPR, see e.g. Compound 26 vs. Compound 27 as well as Compound 30 vs. Compound 31.

Presence of Aib in position 2, e.g. Compound 28 resulted in increased internalization with a relative AUC on 58% of endogenous GIP compared to 39% for Compound 35 and Compound 34. They are however biased with lower internalization compared to endogenous agonist GIP.

TABLE 5
Absolute (AUC = area under the curve) and relative
(%) hGIPR internalization of the tested peptide triple
agonists of GIPR, GLP-1R and GLP-2R with respect to human GIPR.

| | Absolute hGIPR | Relative hGIPR |
|---|---|---|
| Compound no.: | internalization (AUC) | internalization (%) |
| hGIP 1-42 | 37.0 | 100 |
| 1 | 5.3 | 14 |
| 18 | 2.1 | 6 |
| 19 | 8.9 | 24 |
| 20 | 14.2 | 38 |
| 21 | 10.2 | 28 |
| 22 | 7.0 | 19 |
| 23 | 1.2 | 3 |
| 24 | 15.0 | 41 |
| 25 | 13.1 | 35 |
| 26 | 16.4 | 44 |
| 27 | 9.0 | 24 |
| 28 | 21.7 | 59 |
| 30 | 21.0 | 57 |
| 31 | 16.0 | 43 |
| 32 | 6.5 | 18 |
| 33 | 15.8 | 43 |
| 34 | 14.7 | 39 |
| 35 | 14.3 | 38 |

TABLE 6
Absolute (AUC = area under the curve) and relative (%)
hGLP-1R internalization of the tested peptide triple agonists
of GIPR, GLP-1R and GLP-2R with respect to human GLP-1R.

| | Absolute hGLP-1R | Relative hGLP-1R |
|---|---|---|
| Compound no.: | internalization (AUC) | internalization (%) |
| hGLP-1 | 84.1 | 100.0 |
| 30 | 7.3 | 8.6 |
| 31 | 4.9 | 5.8 |
| 32 | 7.0 | 8.3 |
| 33 | 6.4 | 7.7 |
| 34 | 6.0 | 7.1 |
| 35 | 2.0 | 2.4 |
| 20 | 91.8 | 109.14 |
| 21 | 94.7 | 112.59 |
| 22 | 78.81 | 93.70 |
| 28 | 0.96 | 1.14 |

TABLE 7
Absolute (AUC = area under the curve) and relative (%)
hGLP-2R internalization of the tested peptide triple agonists
of GIPR, GLP-1R and GLP-2R with respect to human GLP-2R.

| | Absolute hGLP-2R | Relative hGLP-2R |
|---|---|---|
| Compound no.: | internalization (AUC) | internalization (%) |
| hGLP-1 | 48.6 | 100.0 |
| 30 | 22.7 | 46.8 |
| 31 | 30.6 | 63.0 |
| 32 | 15.0 | 30.8 |
| 33 | 6.4 | 13.2 |
| 34 | 8.0 | 16.5 |
| 35 | 7.6 | 15.7 |
| 20 | 39.4 | 81.0 |
| 21 | 36.3 | 74.6 |
| 22 | 38.0 | 78.2 |
| 28 | 12.0 | 24.8 |

### Example 4: Sequence Overview

K(γGlu-C16) as provided herein should be read as fatty acid C16 is attached to the side chain amino group of a Lysine via a yGlu linker. Thus, the yGlu linker is attached directly to the amino group on the side chain of a Lysine, and the fatty acid is attached directly to the yGlu. The same applies to the other fatty acid and linkers listed in parenthesis herein below.

Compound 1:
(SEQ ID NO: 1)
H-Aib-DGTFISDYSTILDNLAARDFINWLIQTKITK-NH₂,
Compound 2:
(SEQ ID NO: 2)
H-Aib-DGTFISDYSTILDNLAARDFINWLIQTKITK-OH,
Compound 3:
(SEQ ID NO: 3)
H-Aib-DGTFISDYATILDNLAARDFINWLIQTKITK-OH,
Compound 4:
(SEQ ID NO: 4)
H-Aib-DGTFISDYSTILDLLAARDFINWLIQTKITK-NH₂,
Compound 5:
(SEQ ID NO: 5)
H-Aib-DGTFISDYSTILDALAARDFINWLIQTKITK-NH₂,
Compound 6:
(SEQ ID NO: 6)
H-Aib-DGTFISDYSTILDNLAARDFINWLIQTKITK-NH₂,
Compound 7:
(SEQ ID NO: 7)
H-Aib-DGTFISDYSTILENLAARDFINWLIQTKITK-NH₂,
Compound 8:
(SEQ ID NO: 8)
H-Aib-DGTFISDYSTILDNLAAREFINWLIQTKITK-NH₂,
Compound 9:
(SEQ ID NO: 9)
H-Aib-DGTFISDYSTILDNLAARDFIEWLIQTKITK-NH₂,
Compound 10:
(SEQ ID NO: 10)
H-Aib-DGTFISDYSIILDNLAARDFINWLIQTKITK-NH₂,
Compound 11:
(SEQ ID NO: 11)
H-Aib-EGTFISDYSTILDNLAARDFINWLIQTKITK-NH₂,
Compound 12:
(SEQ ID NO: 12)
H-Aib-DGTFISDYST-Aib-LDNLAARDFINWLIQTKITK-NH₂,
Compound 13:
(SEQ ID NO: 13)
H-Aib-DGTFISDYSTELDNLAARDFINWLIQTKITK-NH₂,
Compound 14:
(SEQ ID NO: 14)
H-Aib-DGTFISDYSTILDNLAARDFINWLIQTKGKK-NH₂,
Compound 15:
(SEQ ID NO: 15)
H-Aib-DGTFISDYSTILDNIAARDFINWLIQTKITK-NH₂,
Compound 16:
(SEQ ID NO: 16)
Y-Aib-DGTFISDYSTILDNLAARDFINWLIQTKITK-NH₂,
Compound 17:
(SEQ ID NO: 1)
H-Aib-DGTFISDYSTILDNLAARDFINWLIQTKITK-NH₂,
Compound 18:
(SEQ ID NO: 17)
HADGTFTSDYSTILDNLAAKDFINWLIQTKGKK-OH,
Compound 19:
(SEQ ID NO: 18)
HADGTFTSDYSTILDNLAARDFINWLIQTKGKK-OH,
Compound 20:
(SEQ ID NO: 19)
HADGTFTSDYSIILDKLAARDFINWLIQTKGKKNDWKHNITQ-OH,
Compound 21:
(SEQ ID NO: 20)
HADGTFTSDYSTILD-KLAARDFINWLIQTKGKKNDWKHNITQ-OH,
Compound 22:
(SEQ ID NO: 21)
HADGTFTSDYST-Aib-LDKLAARDFINWLIQTKGKKNDWKHNITQ-
OH,
Compound 23:
(SEQ ID NO: 22)
HADGTFTSDYSTILDKKAARDFINWLIQTKGKKNDWKHNITQ-OH,
Compound 24:
(SEQ ID NO: 23)
HADGTFTSDYSTILDKLAARDFINWLIQTKGKKNDWKHNITQ-OH,
Compound 25:
(SEQ ID NO: 24)
HADGTFTSDYSTILDKLAARDFINWLIQTKGKKNDWKHNITQ-OH,
Compound 26:
(SEQ ID NO: 25)
HADGTFTSDYSTILDKLAARDFINWLIQTKGKKNDWKHNITQK-OH,
Compound 27:
(SEQ ID NO: 26)
HADGTFTSDYST-Aib-LDKLAARDFINWLIQTKGKKNDWKHNITQK-
OH,
Compound 28:
(SEQ ID NO: 27)
H-Aib-DGTFISDYSTILDALAARDFINWLIQTKITK-OH,
Compound 29:
(SEQ ID NO: 28)
H-Aib-DGTFISDYSTILDALAARDFINWLIQTKITK-OH,
Compound 30:
(SEQ ID NO: 45)
HADGTFISDYSTILDKLAARDFINWLIQTKGKK-OH,
Compound 31:
(SEQ ID NO: 46)
HADGTFISDYST-Aib-LDKLAARDFINWLIQTKGKK-OH,
Compound 32:
(SEQ ID NO: 47)
HADGTFISDYSTILDNKAARDFINWLIQTKGKK-OH,
Compound 33:
(SEQ ID NO: 48)
HADGTFISDYSTILDNLAARDFINWLIQTKGKK-OH,
Compound 34:
(SEQ ID NO: 49)
HADGTFISDYSTILDNLAARDFINWLITKGKKK-OH,
Compound 35:
(SEQ ID NO: 50)
HADGTFISDYST-Aib-LDNLAARDFINWLIQTKGKKK-OH
(SEQ ID NO: 29)
NDWKHNITQK
(SEQ ID NO: 30)
NDWK
(SEQ ID NO: 31)
NDWKH
(SEQ ID NO: 32)
NDWKHN
(SEQ ID NO: 33)
NDWKHNI
(SEQ ID NO: 34)
NDWKHNIT
(SEQ ID NO: 35)
NDWKHNITQ
(SEQ ID NO: 36)
AEWKHAITQ
(SEQ ID NO: 37)
YAEGTFISDYSIAMDKIHQQDFVNWLLAQKGKKNDWKHNITQ
(SEQ ID NO: 38)
HDEFERHAEGTFTSDVSSYLEGQAAKEFIAWLVKGRG
(SEQ ID NO: 39)
HAEGTFTSDVSSYLEGQAAKEFIAWLVKGR
(SEQ ID NO: 40)
HAEGTFTSDVSSYLEGQAAKEFIAWLVKGRG
(SEQ ID NO: 41)
HADGSFSDEMNTILDNLAARDFINWLIQTKITD
(SEQ ID NO: 42)
HX₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K
(SEQ ID NO: 43)
HX₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K
(SEQ ID NO: 44)
HX₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K

### REFERENCES

Van der Velden et al. 2021. GLP-1 Val8: A Biased GLP-1R Agonist with Altered Binding Kinetics and Impaired Release of Pancreatic Hormones in Rats

### Items

1. A peptide triple agonist comprising or consisting of the sequence

(SEQ ID NO: 42)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is A or Aib,
X₃ is D or E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, Kor I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
with the proviso that when X₇ is I, then X₂ is Aib, and when X₂ is A, then X₇ is S or T,
wherein said peptide is modified by attaching at least one fatty acid molecule at one or more amino acid residues at any one of positions of the C-terminal region of SEQ ID NO: 42, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).
2. A peptide triple agonist comprising or consisting of the sequence

(SEQ ID NO: 43)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is Aib,
X₃ is D or E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
wherein said peptide is modified by attaching at least one fatty acid molecule at one or more amino acid residues at any one of positions of the C-terminal region of SEQ ID NO: 43, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).
3. A peptide triple agonist comprising or consisting of the sequence

(SEQ ID NO: 44)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J

wherein
X₁ is H or Y
X₂ is A or Aib,
X₃ is D or E,
X₇ is S or T,
X₁₁ is S or A,
X₁₂ is T or I,
X₁₃ is I, Aib or E,
X₁₅ is D or E,
X₁₆ is N, L, A, K or E,
X₁₇ is L, Kor I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
wherein said peptide is modified by attaching at least one fatty acid molecule at one or more amino acid residues at any one of positions of the C-terminal region of SEQ ID NO: 44, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).
4. The peptide triple agonist according to item 1, wherein said C-terminal region of SEQ ID NO: 42 consists of the amino acid residues from position 16 to the C-terminus of SEQ ID NO: 42.
5. The peptide triple agonist according to anyone of the preceding items, wherein said C-terminal region of SEQ ID NO: 42 consists of amino acid residues 16 to 33 of SEQ ID NO: 42.
6. The peptide triple agonist according anyone of the preceding items, wherein said C-terminal region of SEQ ID NO: 42 consists of amino acid residues 16 to 43 of SEQ ID NO: 42.
7. The peptide triple agonist according anyone of the preceding items, wherein said peptide is modified by attaching at least one fatty acid molecule at one or more amino acid residues at any one of positions 16 to 33 of SEQ ID NO: 42 and/or at any one of positions of J (SEQ ID NO: 29).
8. The peptide triple agonist according to anyone of the preceding items, wherein X₂ is Aib, and
   wherein said peptide is modified by attaching at least one fatty acid molecule at the C-terminus of SEQ ID NO: 42.
9. The peptide triple agonist according to anyone of the preceding items, wherein X₂ is Aib, and
   wherein said peptide is modified by attaching at least one fatty acid molecule at the amino acid residue at position 33 of SEQ ID NO: 42.
10. The peptide according to anyone of the preceding items, wherein X₁₆ is N, L, A, or E and
   wherein said peptide is modified by attaching at least one fatty acid molecule at the C-terminus of SEQ ID NO: 42.
11. The peptide according to anyone of the preceding items, wherein X₁₇ is L, or I.
12. The peptide according to anyone of the preceding items, wherein X₂₀ is R.
13. The peptide trial agonist according to anyone of the preceding items, wherein X₇ is T.
14. The peptide trial agonist according to anyone of the preceding items, wherein X₇ is S.
15. The peptide trial agonist according to anyone of the preceding items, wherein X₁₃ is Aib.
16. The peptide triple agonist according to anyone of the preceding items, wherein X₁₆ is N, A or K.
17. The peptide triple agonist according to anyone of the preceding items, wherein X₂₈ is Q.
18. The peptide triple agonist according to anyone of the preceding items, wherein X₂₉ is T.
19. The peptide triple agonist according to anyone of the preceding items, wherein X₃₀ is K.
20. The peptide triple agonist according to anyone of the preceding items, wherein X₂ is Aib, X₃₁ is I, and X₃₂ is T.
21. The peptide triple agonist according to anyone of the preceding items, wherein X₂ is Aib, X₃₁ is G, and X₃₂ is K.
22. The peptide triple agonist according to anyone of the preceding items, wherein X₇ is T or S, X₃₁ is G, and X₃₂ is K.
23. The peptide triple agonist according to anyone of the preceding items, wherein X₇ is T, X₃₁ is G, and X₃₂ is K.
24. The peptide triple agonist according to anyone of the preceding items, wherein J is omitted.
25. The peptide triple agonist according to anyone of the preceding items, wherein J is selected from the group consisting of K, NDWKHNITQ, NDWKHNITQK.
26. The peptide triple agonist according to any one of the preceding items, wherein said peptide is selected from the group consisting of:

(SEQ ID NO: 1)
H-Aib-DGTFISDYSTILDNLAARDFINWLIQTKITK-NH₂,
(SEQ ID NO: 2)
H-Aib-DGTFISDYSTILDNLAARDFINWLIQTKITK-OH,
(SEQ ID NO: 3)
H-Aib-DGTFISDYATILDNLAARDFINWLIQTKITK-OH,
(SEQ ID NO: 4)
H-Aib-DGTFISDYSTILDLLAARDFINWLIQTKITK-NH₂,
(SEQ ID NO: 5)
H-Aib-DGTFISDYSTILDALAARDFINWLIQTKITK-NH₂,
(SEQ ID NO: 6)
H-Aib-DGTFISDYSTILDNLAARDFINWLIQTKITK-NH₂,
(SEQ ID NO: 7)
H-Aib-DGTFISDYSTILENLAARDFINWLIQTKITK-NH₂,
(SEQ ID NO: 8)
H-Aib-DGTFISDYSTILDNLAAREFINWLIQTKITK-NH₂,
(SEQ ID NO: 9)
H-Aib-DGTFISDYSTILDNLAARDFIEWLIQTKITK-NH₂,
(SEQ ID NO: 10)
H-Aib-DGTFISDYSIILDNLAARDFINWLIQTKITK-NH₂,
(SEQ ID NO: 11)
H-Aib-EGTFISDYSTILDNLAARDFINWLIQTKITK-NH₂,
(SEQ ID NO: 12)
H-Aib-DGTFISDYST-Aib-LDNLAARDFINWLIQTKITK-NH₂,
(SEQ ID NO: 13)
H-Aib-DGTFISDYSTELDNLAARDFINWLIQTKITK-NH₂,
(SEQ ID NO: 14)
H-Aib-DGTFISDYSTILDNLAARDFINWLIQTKGKK-NH₂,
(SEQ ID NO: 15)
H-Aib-DGTFISDYSTILDNIAARDFINWLIQTKITK-NH₂,
(SEQ ID NO: 16)
Y-Aib-DGTFISDYSTILDNLAARDFINWLIQTKITK-NH₂,
(SEQ ID NO: 1)
H-Aib-DGTFISDYSTILDNLAARDFINWLIQTKITK-NH₂,
(SEQ ID NO: 17)
HADGTFTSDYSTILDNLAAKDFINWLIQTKGKK-OH,
(SEQ ID NO: 18)
HADGTFTSDYSTILDNLAARDFINWLIQTKGKK-OH,
(SEQ ID NO: 19)
HADGTFTSDYSIILDKLAARDFINWLIQTKGKKNDWKHNITQ-OH,
(SEQ ID NO: 20)
HADGTFTSDYSTILD-KLAARDFINWLIQTKGKKNDWKHNITQ-OH,
(SEQ ID NO: 21)
HADGTFTSDYST-Aib-LDKLAARDFINWLIQTKGKKNDWKHNITQ-
OH,
(SEQ ID NO: 22)
HADGTFTSDYSTILDKKAARDFINWLIQTKGKKNDWKHNITQ-OH,
(SEQ ID NO: 23)
HADGTFTSDYSTILDKLAARDFINWLIQTKGKKNDWKHNITQ-OH,
(SEQ ID NO: 24)
HADGTFTSDYSTILDKLAARDFINWLIQTKGKKNDWKHNITQ-OH,
(SEQ ID NO: 25)
HADGTFTSDYSTILDKLAARDFINWLIQTKGKKNDWKHNITQK-OH,
(SEQ ID NO: 26)
HADGTFTSDYST-Aib-LDKLAARDFINWLIQTKGKKNDWKHNITQK-
OH,
(SEQ ID NO: 27)
H-Aib-DGTFISDYSTILDALAARDFINWLIQTKITK-OH,
(SEQ ID NO: 28)
H-Aib-DGTFISDYSTILDALAARDFINWLIQTKITK-OH,
(SEQ ID NO: 45)
HADGTFISDYSTILDKLAARDFINWLIQTKGKK-OH,
(SEQ ID NO: 46)
HADGTFISDYST-Aib-LDKLAARDFINWLIQTKGKK-OH,
(SEQ ID NO: 47)
HADGTFISDYSTILDNKAARDFINWLIQTKGKK-OH,
(SEQ ID NO: 48)
HADGTFISDYSTILDNLAARDFINWLIQTKGKK-OH,
(SEQ ID NO: 49)
HADGTFISDYSTILDNLAARDFINWLITKGKKK-OH,
and
(SEQ ID NO: 50)
HADGTFISDYST-Aib-LDNLAARDFINWLIQTKGKKK-OH,

or a functional variant thereof, wherein said functional variant comprises 1 or 2 individual amino acid substitutions.
27. The peptide triple agonist according to any one of the preceding items, wherein said peptide is C-terminally amidated (—NH₂) or wherein the C-terminus is a carboxylic acid.
28. The peptide triple agonist according to any one of the preceding items, wherein said peptide is modified by attaching at least one fatty acid molecule at a Lysine (K) at any one of positions of the C-terminal region of SEQ ID NO: 42.
29. The peptide triple agonist according to anyone of the preceding items, wherein said peptide is modified by attaching at least one fatty acid molecule at any one of position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, positon 39, position 40, position 41, position 42 or position 43 of SEQ ID NO: 42.
30. The peptide triple agonist according to anyone of the preceding items, wherein said peptide is modified by attaching at least one fatty acid molecule at any one of position 16, position 17, position 20, position 33, position 34, position 37 or position 43 of SEQ ID NO: 42.
31. The peptide triple agonist according to anyone of items 1, 9 to 11, wherein said peptide is modified by attaching at least one fatty acid molecule at one or more amino acid residues at any one of positions 16, 17, 20 to 43 of SEQ ID NO: 42.
32. The peptide triple agonist according to any one of the preceding items, wherein said fatty acid molecule is a straight-chain fatty acid.
33. The peptide triple agonist according to any one of the preceding items, wherein said fatty acid molecule is a branched fatty acid.
34. The peptide triple agonist according to any one of the preceding items, wherein said fatty acid molecule is a monoacyl fatty acid molecule, comprising one fatty acid.
35. The peptide triple agonist according to any one of the preceding items, wherein said fatty acid molecule is a diacyl fatty acid molecule.
36. The peptide triple agonist according to any one of the preceding items, wherein said fatty acid molecule comprises an acyl group selected from the group consisting of CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20).
37. The peptide triple agonist according to any one of the preceding items, wherein said fatty acid molecule comprises two acyl groups individually selected from the group consisting of HOOC—CH₃(CH₂)₈CO— (decanoyl, C10), HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), and HOOC—CH₃(CH₂)₁₉CO— (19-carboxy-nonadecanoyl, C21).
38. The peptide triple agonist according to any one of the preceding items, wherein said fatty acid molecule is attached to an amino acid residue via a spacer.
39. The peptide triple agonist according to any one of the preceding items, wherein said spacer comprises yGlu.
40. The peptide triple agonist according to any one of the preceding items, wherein said spacer comprises or consists of yGlu or γGlu-yGlu.
41. The peptide triple agonist according to any one of the preceding items, wherein said peptide
   a. activates the human GIPR with a potency (Eₘₐₓ values) which is at least 65% of the efficacy by which native human GIP activates the human GIPR, such as at least 70%, such as at least 75%, such as at least 80%, such as at least 85%, such as at least 90%, such as at least 95% of the efficacy by which native human GIP activates the human GIPR;
   b. activates the human GLP-2R with an efficacy (Eₘₐₓ values) which is at least 65% of the efficacy by which native human GLP-2 activates the human GLP-2R, such as at least 70%, such as at least 75%, such as at least 80%, such as at least 85%, such as at least 90%, such as at least 95% of the efficacy by which native human GLP-2 activates the human GLP-2R;
   c. activates the human GLP-1R with an efficacy (Eₘₐₓ values) which is at least 65% of the efficacy by which native human GLP-1 activates the human GLP-1R, such as at least 70%, such as at least 75%, such as at least 80%, such as at least 85%, such as at least 90%, such as at least 95% of the efficacy by which native human GLP-1 activates the human GLP-1R.
42. The peptide triple agonist according to any one of the preceding items, wherein said peptide
   a. has an EC₅₀ towards the human GIPR of 10 nM or less;
   b. has an EC₅₀ towards the human GLP-2R of 50 nM or less; and
   c. has an EC₅₀ towards the human GLP-1R of 12 nM or less.
43. The peptide triple agonist according to any one of the preceding items, wherein said peptide is selected from the group consisting of:

(SEQ ID NO: 1)
H-Aib-DGTFISDYSTILDNLAARDFINWLIQTKIT-K(C10)-NH₂,
(SEQ ID NO: 2)
H-Aib-DGTFISDYSTILDNLAARDFINWLIQTKIT-K(C10)-OH,
(SEQ ID NO: 3)
H-Aib-DGTFISDYATILDNLAARDFINWLIQTKIT-K(C10)-OH,
(SEQ ID NO: 4)
H-Aib-DGTFISDYSTILDLLAARDFINWLIQTKIT-K(C10)-NH₂,
(SEQ ID NO: 5)
H-Aib-DGTFISDYSTILDALAARDFINWLIQTKIT-K(C10)-NH₂,
(SEQ ID NO: 6)
H-Aib-DGTFISDYSTILDNLAARDFINWLIQTKIT-K(C10)-NH₂,
(SEQ ID NO: 7)
H-Aib-DGTFISDYSTILENLAARDFINWLIQTKIT-K(C10)-NH₂,
(SEQ ID NO: 8)
H-Aib-DGTFISDYSTILDNLAAREFINWLIQTKIT-K(C10)-NH₂,
(SEQ ID NO: 9)
H-Aib-DGTFISDYSTILDNLAARDFIEWLIQTKIT-K(C10)-NH₂,
(SEQ ID NO: 10)
H-Aib-DGTFISDYSIILDNLAARDFINWLIQTKIT-K(C10)-NH₂,
(SEQ ID NO: 11)
H-Aib-EGTFISDYSTILDNLAARDFINWLIQTKIT-K(C10)-NH₂,
(SEQ ID NO: 12)
H-Aib-DGTFISDYST-Aib-LDNLAARDFINWLIQTKIT-K(C10)-NH₂,
(SEQ ID NO: 13)
H-Aib-DGTFISDYSTELDNLAARDFINWLIQTKIT-K(C10)-NH₂,
(SEQ ID NO: 14)
H-Aib-DGTFISDYSTILDNLAARDFINWLIQTKGK-K(C10)-NH₂,
(SEQ ID NO: 15)
H-Aib-DGTFISDYSTILDNIAARDFINWLIQTKIT-K(C10)-NH₂,
(SEQ ID NO: 16)
Y-Aib-DGTFISDYSTILDNLAARDFINWLIQTKIT-K(C10)-NH₂,
(SEQ ID NO: 1)
H-Aib-DGTFISDYSTILDNLAARDFINWLIQTKIT-K(γGlu-C10)-NH₂,
(SEQ ID NO: 17)
HADGTFTSDYSTILDNLAA-K(2xAEEA-γGlu-C18-diacid)-DFINWLIQTKGKK-OH,
(SEQ ID NO: 18)
HADGTFTSDYSTILDNLAARDFINWLIQTKGK-K(γGlu-C18-diacid)-OH,
(SEQ ID NO: 19)
HADGTFTSDYSIILD-K(2xAEEA-γGlu-C18-diacid)-
LAARDFINWLIQTKGKKNDWKHNITQ-OH,
(SEQ ID NO: 20)
HADGTFTSDYSTILD-K(2xAEEA-γGlu-C18-diacid)-
LAARDFINWLIQTKGKKNDWKHNITQ-OH,
(SEQ ID NO: 21)
HADGTFTSDYST-Aib-LD-K(2xAEEA-γGlu-C18-diacid)-
LAARDFINWLIQTKGKKNDWKHNITQ-OH,
SEQ ID NO: 22)
HADGTFTSDYSTILDK-K(2xAEEA-γGlu-C18-diacid)-
AARDFINWLIQTKGKKNDWKHNITQ-OH,
(SEQ ID NO: 23)
HADGTFTSDYSTILDKLAARDFINWLIQTKGK-K(2xAEEA-γGlu-C18-diacid)-
NDWKHNITQ-OH,
(SEQ ID NO: 24)
HADGTFTSDYSTILDKLAARDFINWLIQTKGKKNDW-K(2xAEEA-γGlu-C18-
diacid)-HNITQ-OH,
(SEQ ID NO: 25)
HADGTFTSDYSTILDKLAARDFINWLIQTKGKKNDWKHNITQ-K(2xAEEA-γGlu-
C18-diacid)-OH,
SEQ ID NO: 26)
HADGTFTSDYST-Aib-LDKLAARDFINWLIQTKGKKNDWKHNITQ-K(2xAEEA-
γGlu-C18-diacid)-OH,
(SEQ ID NO: 27)
H-Aib-DGTFISDYSTILDALAARDFINWLIQTKIT-K(2xAEEA-γGlu-C18-diacid)-OH,
(SEQ ID NO: 45)
HADGTFISDYSTILDK-(2xAEEA-γGlu-C18-diacid)-LAARDFINWLIQTKGKK-OH,
(SEQ ID NO: 46)
HADGTFISDYST-Aib-LDK-(2xAEEA-γGlu-C18-diacid)-LAARDFINWLIQTKGKK-
OH,
(SEQ ID NO: 47)
HADGTFISDYSTILDNK-(2xAEEA-γGlu-C18-diacid)-AARDFINWLIQTKGKK-OH,
(SEQ ID NO: 48)
HADGTFISDYSTILDNLAARDFINWLIQTKGKK-(2xAEEA-γGlu-C18-diacid)-OH,
(SEQ ID NO: 49)
HADGTFISDYSTILDNLAARDFINWLIQTKGKKK-(2xAEEA-γGlu-C18-diacid)-OH,
or
(SEQ ID NO: 50)
HADGTFISDYST-Aib-LDNLAARDFINWLIQTKGKKK-(2xAEEA-γGlu-C18-diacid)-
OH,

or a functional variant thereof, wherein said functional variant comprises 1 or 2 individual amino acid substitutions.
44. A peptide triple agonist according to any of the preceding items for use as a medicament.
45. A peptide triple agonist according to any one of the preceding items for use in a method of inhibiting bone resorption and/or stimulating bone formation.
46. A peptide triple agonist according to any one of the preceding items for use in a method of treating a bone disorder.
47. The peptide triple agonist for use according to any one of the preceding items, wherein said bone disorder is selected from the group consisting of osteopenia, osteoporosis, severe osteoporosis, post-menopausal osteoporosis, iodiopathic osteoporosis, osteomalacia, rickets, osteitis fibrosa cystica (OFC) and Paget's disease of bone.
48. A peptide triple agonist according to any one of the preceding items for use in a method of treating iodiopathic osteoporosis.
49. A peptide triple agonist according to any one of the preceding items for use in a method of treating osteoporosis in an individual suffering from Duchenne muscular dystrophy (DMD) or cerebral Palsy.

1.-46. (canceled)

47. A peptide triple agonist comprising or consisting of the sequence
(SEQ ID NO: 42)
X₁X₂X₃GTFX₇SDYX₁₁X₁₂X₁₃LX₁₅X₁₆X₁₇AAX₂₀X₂₁FIX₂₄WLIX₂₈X₂₉X₃₀
X₃₁X₃₂K-J
wherein
X₁ is H or Y
X₂ is A or Aib,
X₃ is D or E,
X₇ is I, S or T,
X₁₁ is S or A,
X₁₂ is T or I,
X₁₃ is I, Aib or E,
X₁₅ is D or E,
X₁₆ is N, L, A, K or E,
X₁₇ is L, K or I,
X₂₀ is R or K,
X₂₁ is D, E or K,
X₂₄ is N or E,
X₂₈ is Q, A or K,
X₂₉ is T, Q or K,
X₃₀ is R or K,
X₃₁ is I or G, and
X₃₂ is T or K,
wherein J is a peptide comprising one or more amino acid residues of the sequence NDWKHNITQK (SEQ ID NO: 29) or is omitted,
with the proviso that when X₇ is I, then X₂ is Aib, and when X₂ is A, then X₇ is S or T,
wherein said peptide is modified by attaching at least one fatty acid molecule at one or more amino acid residues at any one of positions of the C-terminal region of SEQ ID NO: 42, wherein said C-terminal region of SEQ ID NO: 42 consists of the amino acid residues from position 16 to the C-terminus of SEQ ID NO: 42, and
wherein said peptide is an agonist of GIPR (glucose-dependent insulinotropic polypeptide receptor), is an agonist of GLP-2R (glucagon-like peptide-2 receptor) and is an agonist of GLP-1R (glucagon-like peptide-1 receptor).

48. The peptide triple agonist according to claim 47, wherein said peptide is modified by attaching at least one fatty acid molecule at one or more amino acid residues at any one of positions 16 to 33 of SEQ ID NO: 42 and/or at any one of positions of J (SEQ ID NO: 29).

49. The peptide triple agonist according to claim 47, wherein X₂ is Aib, and
wherein said peptide is modified by attaching at least one fatty acid molecule at the C-terminal amino acid of SEQ ID NO: 42, or
wherein X₂ is Aib, and wherein said peptide is modified by attaching at least one fatty acid molecule at the amino acid residue at position 33 of SEQ ID NO: 42.

50. The peptide according to claim 47, wherein X₁₆ is N, L, A, or E and
wherein said peptide is modified by attaching at least one fatty acid molecule at the C-terminal amino acid of SEQ ID NO: 42.

51. The peptide according to claim 47, wherein X₁₇ is L, or I, wherein X₂₀ is R, wherein X₇ is T, wherein X₇ is S, wherein X₁₃ is Aib, wherein X₁₆ is N, A or K, wherein X₂₈ is Q, wherein X₂₉ is T, wherein X₃₀ is K, wherein X₂ is Aib, X₃₁ is I, and X₃₂ is T, wherein X₂ is Aib, X₃₁ is G, and X₃₂ is K, wherein X₇ is T or S, X₃₁ is G, and X₃₂ is K, or wherein X₇ is T, X₃₁ is G, and X₃₂ is K.

52. The peptide triple agonist according to claim 47, wherein J is omitted.

53. The peptide triple agonist according to claim 47, wherein J is selected from the group consisting of K, NDWKHNITQ (SEQ ID NO: 35), NDWKHNITQK (SEQ ID NO: 29), or a functional variant thereof, wherein said functional variant comprises 1 or 2 individual amino acid substitutions.

54. The peptide triple agonist according to claim 47, wherein said peptide is:
(SEQ ID NO: 1)
H-Aib-DGTFISDYSTILDNLAARDFINWLIQTKITK,
(SEQ ID NO: 2)
H-Aib-DGTFISDYSTILDNLAARDFINWLIQTKITK,
(SEQ ID NO: 3)
H-Aib-DGTFISDYATILDNLAARDFINWLIQTKITK,
(SEQ ID NO: 4)
H-Aib-DGTFISDYSTILDLLAARDFINWLIQTKITK,
(SEQ ID NO: 5)
H-Aib-DGTFISDYSTILDALAARDFINWLIQTKITK,
(SEQ ID NO: 6)
H-Aib-DGTFISDYSTILDNLAARDFINWLIQTKITK,
(SEQ ID NO: 7)
H-Aib-DGTFISDYSTILENLAARDFINWLIQTKITK,
(SEQ ID NO: 8)
H-Aib-DGTFISDYSTILDNLAAREFINWLIQTKITK,
(SEQ ID NO: 9)
H-Aib-DGTFISDYSTILDNLAARDFIEWLIQTKITK,
(SEQ ID NO: 10)
H-Aib-DGTFISDYSIILDNLAARDFINWLIQTKITK,
(SEQ ID NO: 11)
H-Aib-EGTFISDYSTILDNLAARDFINWLIQTKITK,
(SEQ ID NO: 12)
H-Aib-DGTFISDYST-Aib-LDNLAARDFINWLIQTKITK,
(SEQ ID NO: 13)
H-Aib-DGTFISDYSTELDNLAARDFINWLIQTKITK,
(SEQ ID NO: 14)
H-Aib-DGTFISDYSTILDNLAARDFINWLIQTKGKK,
(SEQ ID NO: 15)
H-Aib-DGTFISDYSTILDNIAARDFINWLIQTKITK,
(SEQ ID NO: 16)
Y-Aib-DGTFISDYSTILDNLAARDFINWLIQTKITK,
(SEQ ID NO: 1)
H-Aib-DGTFISDYSTILDNLAARDFINWLIQTKITK,
(SEQ ID NO: 17)
HADGTFTSDYSTILDNLAAKDFINWLIQTKGKK,
(SEQ ID NO: 18)
HADGTFTSDYSTILDNLAARDFINWLIQTKGKK,
(SEQ ID NO: 19)
HADGTFTSDYSIILDKLAARDFINWLIQTKGKKNDWKHNITQ,
(SEQ ID NO: 20)
HADGTFTSDYSTILD-KLAARDFINWLIQTKGKKNDWKHNITQ,
(SEQ ID NO: 21)
HADGTFTSDYST-Aib-LDKLAARDFINWLIQTKGKKNDWKHNITQ,
(SEQ ID NO: 22)
HADGTFTSDYSTILDKKAARDFINWLIQTKGKKNDWKHNITQ,
(SEQ ID NO: 23)
HADGTFTSDYSTILDKLAARDFINWLIQTKGKKNDWKHNITQ,
(SEQ ID NO: 24)
HADGTFTSDYSTILDKLAARDFINWLIQTKGKKNDWKHNITQ,
(SEQ ID NO: 25)
HADGTFTSDYSTILDKLAARDFINWLIQTKGKKNDWKHNITQK,
(SEQ ID NO: 26)
HADGTFTSDYST-Aib-LDKLAARDFINWLIQTKGKKNDWKHNITQK,
(SEQ ID NO: 27)
H-Aib-DGTFISDYSTILDALAARDFINWLIQTKITK,
(SEQ ID NO: 45)
HADGTFISDYSTILDKLAARDFINWLIQTKGKK-OH,
(SEQ ID NO: 46)
HADGTFISDYST-Aib-LDKLAARDFINWLIQTKGKK-OH,
(SEQ ID NO: 47)
HADGTFISDYSTILDNKAARDFINWLIQTKGKK-OH,
(SEQ ID NO: 48)
HADGTFISDYSTILDNLAARDFINWLIQTKGKK-OH,
(SEQ ID NO: 49)
HADGTFISDYSTILDNLAARDFINWLIQTKGKKK-OH,
or
(SEQ ID NO: 50)
HADGTFISDYST-Aib-LDNLAARDFINWLIQTKGKKK-OH,
or a functional variant thereof, wherein said functional variant comprises 1 or 2 individual amino acid substitutions.

55. The peptide triple agonist according to claim 47, wherein said peptide is C-terminally amidated (—NH₂) or wherein the C-terminus is a carboxylic acid.

56. The peptide triple agonist according to claim 47, wherein said peptide is modified by attaching at least one fatty acid molecule at a Lysine (K) at one or more amino acid residues at any one of positions 16 to 33 of SEQ ID NO: 42.

57. The peptide triple agonist according to claim 47, wherein said peptide is modified by attaching at least one fatty acid molecule at a Lysine (K) at any one of positions of J (SEQ ID NO: 29).

58. The peptide triple agonist according to claim 47, wherein said fatty acid molecule is a straight-chain fatty acid, or wherein said fatty acid molecule is a branched fatty acid.

59. The peptide triple agonist according to claim 47 wherein said fatty acid molecule is a monoacyl fatty acid molecule, or wherein said fatty acid molecule is a diacyl fatty acid molecule.

60. The peptide triple agonist according to claim 47, wherein said fatty acid molecule comprises an acyl group selected from CH₃(CH₂)₈CO— (capriyl, C10), CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18) and CH₃(CH₂)₁₈CO— (arachidyl, C20) or
wherein said fatty acid molecule comprises two acyl groups individually selected from HOOC—CH3(CH2)8CO— (decanoyl, C10), HOOC—CH3(CH2)10CO— (dodecanoyl, C12), HOOC—CH3(CH2)12CO— (1-tetradecanoyl, C14), HOOC—CH3(CH2)14CO— (hexadecanoyl, C16), HOOC—CH3(CH2)15CO— (15-carboxy-pentadecanoyl, C17), HOOC—CH3(CH2)16CO— (octadecanoyl, C18), HOOC—CH3(CH2)17CO— (17-carboxy-heptadecanoyl, C19), HOOC—CH3(CH2)18CO— (eicosanoyl, C20), and HOOC—CH3(CH2)19CO— (19-carboxy-nonadecanoyl, C21).

61. The peptide triple agonist according to claim 47, wherein said fatty acid molecule is attached to an amino acid residue via a spacer.

62. The peptide triple agonist according to claim 61, wherein said spacer comprises or consists of yGlu or γGlu-yGlu.

63. The peptide triple agonist according to claim 47, wherein said peptide is:
(SEQ ID NO: 1)
H-Aib-DGTFISDYSTILDNLAARDFINWLIQTKIT-K(C10)-NH₂,
(SEQ ID NO: 2)
H-Aib-DGTFISDYSTILDNLAARDFINWLIQTKIT-K(C10)-OH,
(SEQ ID NO: 3)
H-Aib-DGTFISDYATILDNLAARDFINWLIQTKIT-K(C10)-OH,
(SEQ ID NO: 4)
H-Aib-DGTFISDYSTILDLLAARDFINWLIQTKIT-K(C10)-NH₂,
(SEQ ID NO: 5)
H-Aib-DGTFISDYSTILDALAARDFINWLIQTKIT-K(C10)-NH₂,
(SEQ ID NO: 6)
H-Aib-DGTFISDYSTILDNLAARDFINWLIQTKIT-K(C10)-NH₂,
(SEQ ID NO: 7)
H-Aib-DGTFISDYSTILENLAARDFINWLIQTKIT-K(C10)-NH₂,
(SEQ ID NO: 8)
H-Aib-DGTFISDYSTILDNLAAREFINWLIQTKIT-K(C10)-NH₂,
(SEQ ID NO: 9)
H-Aib-DGTFISDYSTILDNLAARDFIEWLIQTKIT-K(C10)-NH₂,
(SEQ ID NO: 10)
H-Aib-DGTFISDYSIILDNLAARDFINWLIQTKIT-K(C10)-NH₂,
(SEQ ID NO: 11)
H-Aib-EGTFISDYSTILDNLAARDFINWLIQTKIT-K(C10)-NH₂,
(SEQ ID NO: 12)
H-Aib-DGTFISDYST-Aib-LDNLAARDFINWLIQTKIT-K(C10)-NH₂,
(SEQ ID NO: 13)
H-Aib-DGTFISDYSTELDNLAARDFINWLIQTKIT-K(C10)-NH₂,
(SEQ ID NO: 14)
H-Aib-DGTFISDYSTILDNLAARDFINWLIQTKGK-K(C10)-NH₂,
(SEQ ID NO: 15)
H-Aib-DGTFISDYSTILDNIAARDFINWLIQTKIT-K(C10)-NH₂,
(SEQ ID NO: 16)
Y-Aib-DGTFISDYSTILDNLAARDFINWLIQTKIT-K(C10)-NH₂,
(SEQ ID NO: 1)
H-Aib-DGTFISDYSTILDNLAARDFINWLIQTKIT-K(γGlu-C10)-NH₂,
(SEQ ID NO: 17)
HADGTFTSDYSTILDNLAA-K(2xAEEA-γGlu-C18-diacid)-DFINWLIQTKGKK-OH,
(SEQ ID NO: 18)
HADGTFTSDYSTILDNLAARDFINWLIQTKGK-K(γGlu-C18-diacid)-OH,
(SEQ ID NO: 19)
HADGTFTSDYSIILD-K(2xAEEA-γGlu-C18-diacid)-
LAARDFINWLIQTKGKKNDWKHNITQ-OH,
(SEQ ID NO: 20)
HADGTFTSDYSTILD-K(2xAEEA-γGlu-C18-diacid)-
LAARDFINWLIQTKGKKNDWKHNITQ-OH,
(SEQ ID NO: 21)
HADGTFTSDYST-Aib-LD-K(2xAEEA-γGlu-C18-diacid)-
LAARDFINWLIQTKGKKNDWKHNITQ-OH,
SEQ ID NO: 22)
HADGTFTSDYSTILDK-K(2xAEEA-γGlu-C18-diacid)-
AARDFINWLIQTKGKKNDWKHNITQ-OH,
(SEQ ID NO: 23)
HADGTFTSDYSTILDKLAARDFINWLIQTKGK-K(2xAEEA-γGlu-C18-diacid)-
NDWKHNITQ-OH,
(SEQ ID NO: 24)
HADGTFTSDYSTILDKLAARDFINWLIQTKGKKNDW-K(2xAEEA-γGlu-C18-
diacid)-HNITQ-OH,
(SEQ ID NO: 25)
HADGTFTSDYSTILDKLAARDFINWLIQTKGKKNDWKHNITQ-K(2xAEEA-γGlu-
C18-diacid)-OH,
SEQ ID NO: 26)
HADGTFTSDYST-Aib-LDKLAARDFINWLIQTKGKKNDWKHNITQ-K(2xAEEA-
γGlu-C18-diacid)-OH,
(SEQ ID NO: 27)
H-Aib-DGTFISDYSTILDALAARDFINWLIQTKIT-K(2xAEEA-γGlu-C18-diacid)-OH,
(SEQ ID NO: 45)
HADGTFISDYSTILDK-(2xAEEA-γGlu-C18-diacid)-LAARDFINWLIQTKGKK-OH,
(SEQ ID NO: 46)
HADGTFISDYST-Aib-LDK-(2xAEEA-γGlu-C18-diacid)-LAARDFINWLIQTKGKK-
OH,
(SEQ ID NO: 47)
HADGTFISDYSTILDNK-(2xAEEA-γGlu-C18-diacid)-AARDFINWLIQTKGKK-OH,
(SEQ ID NO: 48)
HADGTFISDYSTILDNLAARDFINWLIQTKGKK-(2xAEEA-γGlu-C18-diacid)-OH,
(SEQ ID NO: 49)
HADGTFISDYSTILDNLAARDFINWLIQTKGKKK-(2xAEEA-γGlu-C18-diacid)-OH,
or
(SEQ ID NO: 50)
HADGTFISDYST-Aib-LDNLAARDFINWLIQTKGKKK-(2xAEEA-γGlu-C18-diacid)-
OH,
or a functional variant thereof, wherein said functional variant comprises 1 or 2 individual amino acid substitutions.

64. A method for treatment of a bone disorder comprising administration of a therapeutically effective amount of a peptide triple agonist according to claim 47 to an individual in need thereof.

65. The method according to claim 64, wherein said bone disorder is osteopenia, osteoporosis, severe osteoporosis, post-menopausal osteoporosis, idiopathic osteoporosis, osteomalacia, rickets, osteitis fibrosa cystica (OFC) or Paget's disease of bone.

66. The method according to claim 65, wherein said bone disorder is osteoporosis in an individual suffering from Duchenne muscular dystrophy (DMD) or cerebral Palsy.
